(19) United States
(12) Patent Application Publication
(10) Pub. No.: US 2016/0235928 A1
(43) Pub. Date: Aug. 18, 2016

(54) INJECTION NEEDLE AND DEVICE

(71) Applicant: ChronTech Pharma AB, Huddinge (SE)

(72) Inventors: Matti Sällberg, Stockholm (SE); Lars Frelin, Alvsjo (SE)

(21) Appl. No.: 14/993,930

(22) Filed: Jan. 12, 2016

Related U.S. Application Data
(63) Continuation of application No. 13/514,269, filed on Jun. 6, 2012, now abandoned.
(60) Provisional application No. 61/287,160, filed on Dec. 16, 2009.
(60) Provisional application No. 61/292,374, filed on Jan. 5, 2010.

(51) Int. Cl. A61M 5/32

(52) U.S. Cl. CPC A61M 5/3298; A61M 5/3291; A61M 5/32; A61M 2202/206

(57) ABSTRACT

A needle device for the delivery of therapeutic material into tissue comprising a connection to a pressure generation element, a lumen adapted for the passage of a therapeutic material, and a needle barrel, wherein each needle barrel comprises an opening adapted to control and deliver a pressure transmitted from the pressure generation element into a tissue to cause an increase in the permeability of a cell membrane to the therapeutic material.

### CROSS-REFERENCE TO RELATED APPLICATIONS

This application claims the benefit and priority to U.S. application Ser. No. 13/514,269, filed Jun. 6, 2012, which, in turn, is a U.S. National Phase Application of PCT International Application Number PCT/IB2010/003399, filed on Dec. 14, 2010, designating the United States of America and published in the English language, which is an International Application of and claims the benefit of priority to U.S. Provisional Application No. 61/287,160, filed on Dec. 16, 2009, and U.S. Provisional Application No. 61/292,374, filed on Jan. 5, 2010. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled TRIPEP104WO.TXT, created Dec. 14, 2010, which is 146 KB in size. The information in the electronic format of the Sequence Listing is incorporated herein by reference in its entirety.

### FIELD OF THE INVENTION

Aspects of the embodiments disclosed herein relate generally to devices and methods for the delivery and uptake of therapeutic material (e.g., chemicals, compounds, proteins and nucleic acids) by tissue of a subject (e.g. a human). Preferred embodiments concern devices and methods for the delivery of genetic material or nucleic acids including, but not limited to, DNA, RNA, and modified nucleic acids into a plurality of cells, preferably animal cells, such as human cells.

### BACKGROUND OF THE INVENTION

The delivery of therapeutic material, such as genetic material, into tissue has a wide range of useful applications including vaccination, replacement of a defective gene, DNA immunization, introduction of an immunogen, anti-sense therapy, and miRNA, RNAi, aptamer, or siRNA therapy. For instance, nucleic acids, such as DNA, for example, can be injected into tissue, wherein the nucleic acids are taken up by the surrounding cells albeit inefficiently. DNA introduced in this manner will produce the protein that the DNA encodes. The successful delivery of nucleic acids into tissue and the uptake of the nucleic acids by the cells is difficult, especially when significant amounts of protein expression are desired (e.g., as is desired for DNA-based vaccination). Conventional injection of genetic material into tissue generally results in poor uptake by the cells and low levels of protein expression, if any at all.

Various methods have been developed to improve delivery and to increase expression of genetic material that is introduced into tissue. For example, researchers have developed electroporation systems to enhance the uptake of DNA and other therapeutic material that is injected into muscles, organs and other tissues (see e.g., U.S. Pat. No. 6,610,044 and U.S. Pat. No. 6,132,419, herein expressly incorporated by reference in their entireties). Electroporation systems generally involve application of an electric field shortly after or simultaneous with the introduction of the DNA at the tissue around and/or through the site of the injection. The electric fields are applied to make the walls of cells sufficiently permeable to permit molecules the size of nucleic acids to enter. Electroporation systems are costly, and require considerable training to administer not mention that patients find the procedure to be painful. Electroporation systems are also not very portable. The complex control circuitry and the need for a reliable external power source make these systems unsuitable for use in remote settings (e.g., a battlefield or developing countries) or in situations where rapid access to DNA vaccination would be needed (e.g., a pandemic viral outbreak).

Intravascular administration approaches have also been developed to deliver therapeutic agents to animals (see e.g., U.S. Pat. Nos. 6,379,966; 6,897,068; 7,015,040; 7,214,369; 7,473,419; and 7,589,059, all of which are hereby expressly incorporated by reference in their entireties). Intravascular administration can be very difficult to implement in practice; however, requiring skilled clinicians and, if performed incorrectly, the procedure can lead to punctured blood vessels, hematomas, and the development of internal blood clots, which could lead to an embolism. Furthermore, the intravascular administration approach can produce a wide dispersion of the introduced therapeutic agent (e.g., nucleic acid and protein), which is undesirable when trying to encourage the body to mount an immune response to the delivered agent. Accordingly, there remains a need for devices and methods that facilitate the delivery and uptake of therapeutic molecules such as nucleic acids and proteins.

### SUMMARY OF THE INVENTION

Disclosed herein are devices and methods that are configured to deliver a therapeutic agent (e.g. a chemical, a compound, a chemotherapeutic agent, a protein, a nucleic acid, such as DNA, RNA, other natural nucleic acid, a modified nucleic acid, or a DNA or nucleic acid aptamer) into tissue, whereby said agent can be taken up by cells in the tissue surrounding the injection site and, the agent is expressed so as to provide a therapeutic or cosmetic benefit. In additional embodiments, one or more of the needles and/or devices described herein are used to administer cell populations (e.g., regenerative cells, stem cells, progenitor cells, or a mixture thereof) to effectuate therapeutic and/or cosmetic benefit. In these embodiments, the cells are introduced into tissue (e.g., fatty tissue of the breast, heart, kidney, bone, skin, fat tissue, intervertebral discs) of a subject in need thereof to promote therapeutic or cosmetic benefit (e.g., to facilitate or effectuate breast reconstruction, ameliorate an ischemic region, repair degenerative discs, promote bone repair, promote wound healing, or to ameliorate wrinkles or pock marks on the skin).

Accordingly, aspects of the invention concern a needle that is configured for delivery of a therapeutic agent (e.g. a cell population, such as a cell population comprising stem cells, chemical, a compound, a chemotherapeutic agent, a protein, a nucleic acid, such as DNA, RNA, other natural nucleic acid, a modified nucleic acid, or a DNA or nucleic acid aptamer), wherein said needle comprises a closed or open end and a plurality of apertures that extend along the length of the needle. The needle can be blunt-ended or can have a beveled, pointed, or sharp end. The needle can be made to a variety of gauges (e.g., at least, equal to or greater than 7, 8, 9, 10, 11, 12, 13, 14, 15, 16, 17, 18, 19, 20, 21, 22, 23, 24, 25, 26, 27, 28, 29, 30, 31, 32, 33, or 34 gauge). Preferably, the needle is of a gauge that is greater than or equal to 20 (e.g., greater than or equal to 20, 21, 22, 23, 24, 25, 26, 27, 28, 29, 30, 31, 32, 33, or 34 gauge) and more preferably, the needle is of a gauge that is greater than or equal to 23 (e.g., 23, 24, 25, 26, 27, 28, 29, 30, 31, 32, 33, or 34 gauge) and most preferably, the needle is of a gauge that is greater than or equal to 25 (e.g., 25, 26, 27, 28, 29, 30, 31, 32, 33, or 34 gauge). In some embodiments, the apertures are not located at or near the tip of the needle. For example, the apertures can be located at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 2 cm, 3 cm, 4 cm, or more apart from the tip of the needle. In some embodiments, the needles do not include any apertures at or near the tip of the needle.

The length of the needle(s) can vary according to the type of delivery desired. In order to target specific cells in the skin or particular tissues, for example, the preferred target depth depends on the particular cell or tissue being targeted and the thickness of the skin of the particular subject (e.g., to target the Langerhan's cells in the dermal space of human skin, it is desired that the delivery encompass, at least, in part, the epidermal tissue depth typically ranging from about 0.025 mm to about 0.2 mm in humans). Accordingly, in embodiments, wherein delivery to Langerhan's cells is desired, needle lengths can be between about 0.025 mm to about 0.2 mm. In some embodiments, it is desired that the therapeutic agents are delivered at a targeted depth just under the stratum corneum and encompassing the epidermis and upper dermis (e.g., in these embodiments preferred needle lengths include between about 0.025 mm to about 2.5 mm) In other embodiments, the therapeutic agents are delivered into the muscle tissue or adipose tissue (e.g., in these embodiments, it is desired that the preferred needle lengths include between about 0.5 cm to about 15 cm). Accordingly, aspects of the invention concern devices that comprise one or more needles and uses thereof, wherein the length of the needle(s) is greater than, equal to, less than or any number in between about 0.025 mm, 0.05 mm, 0.075 mm, 0.1 mm, 0.2 mm, 0.3 mm, 0.4 mm, 0.5 mm, 0.6 mm, 0.7 mm, 0.8 mm, 0.9 mm, 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 125 mm, 150 mm, 175 mm, 200 mm, 225 mm, 250 mm, 275 mm, 300 mm, 325 mm, 350 mm, 375 mm, 400 mm, 425 mm, 450 mm, 475 mm, 500 mm, 525 mm, 550 mm, 575 mm, 600 mm, 625 mm, 650 mm, 675 mm, 700 mm, 725 mm, 750 mm, 775 mm, 800 mm, 825 mm, 850 mm, 875 mm, 900 mm, 925 mm, 950 mm, 975 mm, 1 cm, 1.25 cm, 1.5 cm, 2.0 cm, 2.25 cm, 2.5 cm, 2.75 cm, 3.0 cm, 3.25 cm, 3.5 cm, 3.75 cm, 4.0 cm, 4.25 cm, 4.5 cm, 4.75 cm, 5.0 cm, 5.25 cm, 5.5 cm, 5.75 cm, 6.0 cm, 6.25 cm, 6.5 cm, 6.75 cm, 7.0 cm, 7.25 cm, 7.5 cm, 7.75 cm, 8.0 cm, 8.25 cm, 8.5 cm, 8.75 cm, 9.0 cm, 9.25 cm, 9.5 cm, 9.75 cm, 10.0 cm, 10.25 cm, 10.5 cm, 10.75 cm, 11.0 cm, 11.25 cm, 11.5 cm, 11.75 cm, 12.0 cm, 12.25 cm, 12.5 cm, 12.75 cm, 13.0 cm, 13.25 cm, 13.5 cm, 13.75 cm, 14.0 cm, 15.25 cm, 14.5 cm, 14.75 cm, or 15 cm.

The needle(s) can include a plurality of apertures of a variety of sizes and shapes (e.g., oval, circular, slit, or ovoid shape), which can be produced by machine cutting or laser. The needle can comprise, for example, greater than or equal to 2, 3, 4, 5, 6, 7, 8, 9, 10, 11, 12, 13, 14 or 15 apertures and said apertures can be evenly spaced along the length of the needle, grouped in one area (e.g., spaced in a first or a second zone of the needle, such as, wherein the two zones are demarcated by the two sides opposing the middle point of the length of the needle) or said apertures can be along the length of the needle), or unevenly spaced along the length of the needle. The needle(s) can have a closed or open end but a closed end is preferred, as such a design is configured to increase the pressure of delivery when small diameter apertures (e.g., a size equal to or less than 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0 mm in its widest portion) are employed. The needle(s) can be composed of surgical steel or stainless steel or a metal alloy (e.g., consisting essentially of at least about 52% Ni and at least about 48% Ti).

The needle(s) can also comprise a fitting connector or a needle hub, which may comprise a sleeve with an internal thread. The fitting connector or needle hub is configured to attach the needle to the syringe or vessel containing the agent to be introduced. In some embodiments, the sleeve forms the attachment means and can be screwed onto an outer thread on an attachment part of a syringe. The fitting connectors or needle hubs can also comprise a press-on assembly, a snap-on assembly, or a Luer Taper connection, such as a Luer Lok or Luer Slip connection or a butterfly connector.

The aforementioned needle(s) can be attached to one or more syringe barrels (e.g., permanently affixed or removably attached) and said syringe barrels or the device may contain the therapeutic agent that is to be delivered (e.g., the needle(s) and attached syringe may be pre-loaded with a therapeutic agent, such as a nucleic acid, protein, or cell population for a single-use application). The syringe barrels can be of a variety of sizes (e.g., 0.3 cc-100 cc or more). That is the syringe barrels can be greater than or equal to or any number in between 0.1, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 cc size. The syringe barrels can be constructed from a variety of materials (e.g., metal, plastic, nylon, polyethylene, glass).

The aforementioned needle(s) can be attached to one or more devices that facilitate delivery of therapeutic molecules or agents to tissue, including but not limited to gene guns, electroporation systems, and microneedle devices. The injection needle(s) described herein can be modified for use with existing technologies, including gene gun delivery systems (see e.g., U.S. Pat. Nos. 5,036,006; 5,240,855; and 5,702,384, the disclosures of which are hereby expressly incorporated by reference in their entireties), delivery systems using electroporation (see e.g., U.S. Pat. Nos. 6,610,044 and 5,273,525, the disclosures of which are hereby expressly incorporated by reference in their entireties) and microneedle delivery systems (see e.g., U.S. Pat. Nos. 6,960,193; 6,623,457; 6,334,856; 5,457,041; 5,527,288; 5,697,901; 6,440,096; 6,743,211; and 7,226,439, the disclosures of which are hereby expressly incorporated by reference in their entireties).

As mentioned above, the syringes comprising the needle(s) described herein may also contain a variety of therapeutic agents (e.g. a cell population, such as a cell population comprising stem cells, chemical, a compound, a chemotherapeutic agent, a protein, a nucleic acid, such as DNA, RNA, other natural nucleic acid, a modified nucleic acid, or a DNA or nucleic acid aptamer). In some embodiments, the syringe comprising one or more of the needle(s) described herein comprises a DNA that encodes an immunogen (preferably a viral antigen, such as hepatitis C virus (HCV), hepatitis B virus (HBV), human immunodeficiency virus (HIV), influenza, Japanese encephalitis virus (JEV), human papilloma virus (HPV), or a parasite antigen, such as a malaria antigen, or a plant antigen, such as birch antigen, or a bacterial antigen, such as a staphylococcal or anthrax antigen, or a tumor antigen). In some embodiments, the syringe comprising one or more of the needles described herein comprises one or more of the aforementioned DNAs pre-loaded (e.g., a pre-loaded, single use syringe with coupled needle(s) containing a measured dose of delivered agent).

In some embodiments, the therapeutic agent that is delivered or contained in a syringe, needle, or injection device as described herein comprises a natural nucleic acid and in other embodiments, the therapeutic agent that is delivered or contained in a syringe, needle, or injection device as described herein comprises an unnatural nucleic acid (e.g., containing an artificial nucleotide or spacer). Natural nucleic acids that can be used as the therapeutic agent that is delivered or contained in a syringe or injection device as described herein comprise a deoxyribose- or ribose-phosphate backbone. An artificial or synthetic polynucleotide that can be used as the therapeutic agent that is delivered or contained in a syringe, needle, or injection device as described herein comprise any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases that may be included in one or more embodiments described herein include purines and pyrimidines, which further include the natural compounds adenine, thymine, guanine, cytosine, uracil, inosine, and natural analogs. Synthetic derivatives of purines and pyrimidines that may be included in one or more embodiments described herein include, but are not limited to, modifications which place new reactive groups such as, but not limited to, amines, alcohols, thiols, carboxylates, and alkylhalides. The term “base,” as used herein, encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

The therapeutic agent that is delivered or contained in a syringe, needle, or injection device as described herein can comprise DNA, which may be in the form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. The therapeutic agent that is delivered or contained in a syringe, needle, or injection device as described herein can also comprise an anti-sense polynucleotide that is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15 to 50 base pairs and preferably 21 to 25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids. These polynucleotides can be delivered to a cell to express an exogenous nucleotide sequence, to inhibit, eliminate, augment, or alter expression of an endogenous nucleotide sequence, or to express a specific physiological characteristic not naturally associated with the cell. Polynucleotides may be coded to express a whole or partial protein, or may be anti-sense. The delivered polynucleotide can stay within the cytoplasm or nucleus apart from the endogenous genetic material. Alternatively, the polymer could recombine (become a part of) the endogenous genetic material. For example, the therapeutic agent that is delivered or contained in a syringe or injection device as described herein can comprise a DNA that can insert itself into chromosomal DNA by either homologous or non-homologous recombination.

The therapeutic agent that is delivered or contained in a syringe, needle, or injection device as described herein can also comprise an RNA inhibitor, which is any nucleic acid or nucleic acid analog containing a sequence whose presence or expression in a cell causes the degradation of or inhibits the function or translation of a specific cellular RNA, usually a mRNA, in a sequence-specific manner. An RNA inhibitor may also inhibit the transcription of a gene into RNA Inhibition of RNA can effectively inhibit expression of a gene from which the RNA is transcribed. RNA inhibitors include, but are not limited to, siRNA, interfering RNA or RNAi, dsRNA, RNA Polymerase III transcribed DNAs, ribozymes, and antisense nucleic acid, which may be RNA, DNA, or an artificial nucleic acid. SiRNA can comprise a double stranded structure typically containing 15 50 base pairs and preferably 21 25 base pairs and having a nucleotide sequence identical or nearly identical to an expressed target gene or RNA within the cell. Antisense polynucleotides can include, but are not limited to: morpholinos, 2′-O-methyl polynucleotides, DNA, RNA and the like. RNA polymerase III transcribed DNAs can contain promoters, such as the U6 promoter. These DNAs can be transcribed to produce small hairpin RNAs in the cell that can function as siRNA or linear RNAs that can function as antisense RNA. The RNA inhibitor may be polymerized in vitro, recombinant RNA, contain chimeric sequences, or derivatives of these groups. The RNA inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

The therapeutic agent that is delivered or contained in a syringe, needle, or injection device as described herein can also include a nucleic acid that is incorporated into a vector (e.g., an expression vector). Vectors are polynucleic molecules originating from a virus, a plasmid, or the cell of a higher organism into which another nucleic fragment of appropriate size can be integrated; vectors typically introduce foreign DNA into host cells, where it can be reproduced. Examples are plasmids, cosmids, and yeast artificial chromosomes; vectors are often recombinant molecules containing DNA sequences from several sources. A vector includes a viral vector: for example, adenovirus; DNA; adenoassociated viral vectors (AAV) which are derived from adenoassociated viruses and are smaller than adenoviruses; and retrovirus (any virus in the family Retroviridae that has RNA as its nucleic acid and uses the enzyme reverse transcriptase to copy its genome into the DNA of the host cell's chromosome; examples include VSV G and retroviruses that contain components of lentivirus including HIV type viruses). As used herein, term “vector” refers any DNA molecule that could include associate molecules to transfer DNA sequences into a cell for expression. Examples include naked DNA, non-viral DNA complexes (e.g. DNA plus polymers [cationic or anionic], DNA plus transfection enhancing compounds, and DNA plus amphipathic compounds) and viral particles.

The therapeutic agent that is delivered or contained in a syringe, needle, or injection device as described herein can also comprise one or more compounds that enhance the uptake of the therapeutic agent (e.g., a nucleic acid as described herein). The therapeutic agent that is delivered or contained in a syringe, needle, or injection device as described herein can comprise a polymer, for example, which is a molecule built up by repetitive bonding together of smaller units called monomers. The term “polymer” can include both oligomers, which have two to about 80 monomers and polymers having more than 80 monomers. The polymer can be linear, branched network, star, comb, or ladder types of polymer. The polymer can be a homopolymer in which a single monomer is used or can be copolymer in which two or more monomers are used. Types of copolymers include alternating, random, block and graft.

The therapeutic agent that is delivered or contained in a syringe, needle, or injection device as described herein can also comprise a nucleic acid-polycation complex. Cationic proteins like histones and protamines or synthetic polymers like polylysine, polyarginine, polyornithine, DEAE dextran, polybrene, and polyethylenimine are effective intracellular delivery agents. A polycation is a polymer containing a net positive charge, for example poly-L-lysine hydrobromide. The polycation can contain monomer units that are charge positive, charge neutral, or charge negative, however, the net charge of the polymer is desirably positive. The term “polycation” also can refer to a non-polymeric molecule that contains two or more positive charges. A polyanion is a polymer containing a net negative charge, for example polyglutamic acid. The polyanion can contain monomer units that are charge negative, charge neutral, or charge positive, however, the net charge on the polymer must be negative. The term “polyanion” can also refer to a non-polymeric molecule that contains two or more negative charges. The term “polyion” includes polycation, polyanion, zwitterionic polymers, and neutral polymers that contain equal amounts of anions and cations. The term “zwitterionic” refers to the product (salt) of the reaction between an acidic group and a basic group that are part of the same molecule. Salts are ionic compounds that dissociate into cations and anions when dissolved in solution. Salts increase the ionic strength of a solution, and consequently decrease interactions between nucleic acids with other cations.

Accordingly, some embodiments concern a device that comprises a plurality of the aforementioned needles, which are arranged or configured to deliver a therapeutic agent to a targeted tissue. Aspects of the invention concern an injection device including a plurality of any one of the aforementioned needle barrels, e.g., each needle barrel comprises a plurality of apertures that extend along the length of the needle or are present within distinct zones of said needle and a device containing an agent (e.g. a cell population, such as a cell population comprising stem cells, chemical, a compound, a chemotherapeutic agent, a protein, a nucleic acid, such as DNA, RNA, other natural nucleic acid, a modified nucleic acid, or a DNA or nucleic acid aptamer) connected thereto. In some embodiments, the agent is delivered through the proximal end of the injection device by a syringe and the agent is delivered to the targeted tissue through a plurality of apertures disposed on the distal ends of the needle barrels. In other embodiments, the end of the apertures can be disposed on the proximal ends of the needles barrels.

Preferably, a plurality of needles of any one or more of the design features above are provided on an injection device. Embodiments described herein also include a cannula that comprises a plurality of needles configured as described above. That is, in some embodiments the injection device and/or cannula can comprise, consist, or consist essentially of 2, 3, 4, 5, 6, 7, 8, 9, or 10 needles. The needles can be of the same size and length or can be of different sizes and lengths. Each needle in embodiments that have more than one needle can have a plurality of apertures, which can be in a first or second zone, as described above, or both (e.g., along the length of the band). Injection devices and/or cannulas that comprise, consist, or consist essentially of 2, 3, 4, 5, 6, 7, 8, 9, or 10 needles can be configured such that at least two needles have a different amount of apertures and/or different sizes of apertures and/or different shapes of apertures and/or different positions of apertures. That is, in some embodiments, one needle or a plurality of needles has apertures in a first zone proximal to a closed end of the barrel and one needle or a plurality of needles that has apertures in a second zone that is distal to a closed end of the needle barrel. Additionally, some embodiments may have a first needle or a first plurality of needles with apertures that are smaller or substantially smaller (e.g., a size equal to, greater than or less than 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.0, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, or 4.0 mm in its widest portion) than a second needle or a second plurality of needles.

More embodiments concern the injection devices, cannulas, and needles described above containing or comprising a fluid containing an agent, as described herein (e.g., a medicinal compound, chemical, nucleic acid, in particular, DNA). In some embodiments, the injection devices, cannulas, and needles described herein are for single use. That is, some embodiments comprise one or more of the needle designs described herein joined to a receptacle (preferably a sterile container, such as a sterilized syringe) that comprises a single application or dose of delivered agent (e.g., medicinal compound, chemical, nucleic acid, in particular DNA). Accordingly, a single application or device can be conveniently packaged and provided to medical practitioners or end-consumers (e.g. subjects), which can administer said agent at an appropriate site and, following administration, the used injection device, needle, or cannula comprising a plurality of needles can be appropriately discarded. Methods of making and using the aforementioned devices to, for example, methods of inducing an immune response to a desired antigen, are also embodiments.

In some embodiments, the needle device is not configured to apply an electric field shortly after or simultaneous with the introduction of the therapeutic material (e.g., DNA) at the tissue around and/or through the site of the injection. For example, the needle device may not include a voltage source coupled to the device and configured to apply an electric field to the tissue at or near the site of injection.

Some embodiments disclosed herein include a method of delivering a therapeutic material to a subject in need thereof, where the therapeutic material is administered using any of the injection devices disclosed herein. The therapeutic material may be any of those materials disclosed herein. In some embodiments, the method includes delivering the therapeutic material at a predetermined rate. The predetermined rate, in some embodiments, may be at least 0.1 mL/s, 0.3 mL/s, 0.5 mL/s, 0.8 mL/s, 0.9 mL/s, 1.0 mL/s, 1.1 mL/s, 1.2 mL/s, 1.3, mL/s, 1.4 mL/s, 1.5 mL/s, 2.0 mL/s, or 3.0 mL/s The predetermined rate, in some embodiments, may be no more than 20.0 mL/s, 10.0 mL/s, 7 mL/s, 6 mL/s, 5 mL/s, 4 mL/s, 3 mL/s, or 2 mL/s. In some embodiments, the method may also include maintaining the one ore more needles inserted within the tissue for at least a predetermined time after injecting the therapeutic material but before withdrawing the one or more needles. The one or more needles may be maintained in the tissue, for example, at least, greater than or equal to 1 s, 2 s, 3 s, 4 s, 5 s, or more after injecting the therapeutic material but before withdrawing the one or more needles. In some embodiments, the needles and any of the devices described herein can be affixed to the body of a subject for greater periods of time so as to allow for a long term delivery of a therapeutic agent (e.g., delivery for at least, greater than or equal to 1, 2, 3, 4, 5, 6, 7, 8, 9, 10, 11, 12, 13, or 14 days) and such needles and devices can be affixed to miniature pumps so as to administer small amounts of therapeutic material (e.g. a cell population, such as a cell population comprising stem cells, chemical, a compound, a chemotherapeutic agent, a protein, a nucleic acid, such as DNA, RNA, other natural nucleic acid, a modified nucleic acid, or a DNA or nucleic acid aptamer), to said subjects over an extended period of time.

Preferred aspects of the invention concern a hypodermic needle assembly comprising a needle that comprises a lumen adapted for the passage of a therapeutic material and a needle barrel that comprises a plurality of apertures on the length of the barrel, wherein said needle barrel has a closed-end; and a connector configured to join said needle to a pressure generation element. In some embodiments, the hypodermic needle assembly above comprises a plurality of said needles and in some embodiments, the hypodermic needle assembly comprises a circular, diamond, or ovoid array of said needles. Preferably, the hypodermic needle assembly is designed such that the plurality of said needles is configured such that the apertures on the needle barrels face each other but in some embodiments, the hypodermic needle assembly has a plurality of said needles that is configured such that the apertures on the needle barrels face away from each other. In some embodiments, the hypodermic needle assembly further comprises a pressure generation element joined to said hypodermic needle assembly and this pressure generation element can be a syringe. The hypodermic needle assemblies above of can have apertures that have a diameter of about 10 nm-4 mm, 0.01 mm-4 mm, 0.1 mm-4 mm, 1.0 mm-4 mm, 1.5 mm-4 mm, 2.0 mm-4 mm, or 3.0 mm-4 mm.

In some embodiments, the hypodermic needle assemblies above comprise a single syringe joined to at least three of said needles. In some embodiments, the at least three of said needles are between about 2 and about 10 mm apart. In other embodiments, the hypodermic needle assemblies above can comprise a single syringe joined to at least four hypodermic needles. In some embodiments, the hypodermic needle assembly has at least four hypodermic needles that are between about 3 and about 6 mm apart. A single use hypodermic delivery device is also an embodiment and such devices preferably comprise a plurality of needles attached to at least one syringe, wherein the needles comprise a plurality of apertures distributed along the barrel of said needles and a closed end; and said at least one syringe comprises a single dose of a therapeutic agent. In some embodiments, the therapeutic agent in the hypodermic delivery device is a nucleic acid. The therapeutic agent can be a DNA that encodes a protein. In some embodiments, the hypodermic delivery device above comprises a single syringe joined to at least three hypodermic needles and in some embodiments, the at least three hypodermic needles are between about 2 and about 10 mm apart. In other embodiments, the hypodermic delivery device above comprises a single syringe joined to at least four needles and in some embodiments, the at least four hypodermic needles are between about 3 and about 6 mm apart.

Aspects of the invention also include methods of making and using the aforementioned devices. By one approach, some of the devices described herein are used to deliver a therapeutic agent to a subject and said methods are practiced by providing one of the delivery devices described herein, inserting the needles of said device into a tissue of a subject; and displacing the therapeutic agent from the syringe through the needles and into the tissue. In some embodiments, the therapeutic agent is a nucleic acid, the nucleic acid can encode an antigen, such as a viral antigen, preferably, a hepatitis antigen such as an HCV or HBV antigen such that some of the delivery devices described herein can be used for the purposes of inducing an immune response in a subject to an antigen that is delivered by said device.

Additional embodiments include a hypodermic needle device for the delivery of therapeutic material into tissue, the device comprising a connection to a pressure generation element; a lumen adapted for the passage of a therapeutic material; and a needle barrel, wherein the needle barrel comprises a plurality of apertures that extend along the length of the barrel. In some embodiments, the therapeutic material comprises a nucleic acid, a polypeptide, a carbohydrate, a steroid, a cell population, a chemical or an immunogen. In some embodiments, the therapeutic agent induces the immune system. The tissue can be skeletal muscle, dermal tissue, or adipose tissue, for example. Preferably, the pressure generation element comprises a syringe and the pressure generation element can transmit a pressure of 0.1 kilopascals or greater, 1.0 kilopascals or greater, 10 kilopascals or greater, 100 kilopascals or greater, 150 kilopascals or greater, or 200 kilopascals or greater into the tissue. In some embodiments the aperture(s) along the needle barrel have a diameter of about 10 nm-4 mm, 0.01 mm-4 mm, 0.1 mm-4 mm, 1.0 mm-4 mm, 1.5 mm-4 mm, 2.0 mm-4 mm, or 3.0 mm-4 mm The needle barrel can be adapted to transmit an electric current and the device can further comprises an electrode adapted to transmit an electromagnetic field. In some embodiments, the therapeutic agent enters a cell and in others it remains extracellular. In some embodiments, the pressure is transmitted using a fluid medium or a gas medium. In some embodiments, the nucleic acid comprises a sequence from a hepatitis virus such as a hepatitis B antigen (HBV), such as HBcAg, or a hepatitis C virus (HCV) antigen, such as NS3/4A, or a combination thereof such as HBcAg from an HBV virus that infects stork or heron joined to NS3/4A. In other embodiments, the nucleic acid comprises a sequence from a human simian virus antigen. Preferably, the nucleic acid comprises a sequence encoding an antigen capable of generating a proliferative T cell response and in some embodiments, the nucleic acid comprises a sequence from a human immunodeficiency virus.

Additional embodiments include, a hypodermic needle system for the delivery of therapeutic material into tissue comprising a therapeutic material pressure generation element; an array of needle barrels coupled to the pressure generation element; wherein at least one of the needle barrels in the array comprises a plurality of apertures adapted to deliver a pressure transmitted from the pressure generation element into a tissue to cause an increase in the permeability of a cell membrane, and at least one of the needle barrels in the array is adapted for the passage of the therapeutic material. In some embodiments, the therapeutic material comprises a nucleic acid, a polypeptide, a carbohydrate, a steroid, a cell population, a chemical or an immunogen. In some embodiments, the therapeutic agent induces the immune system. The tissue can be skeletal muscle, dermal tissue, or adipose tissue, for example. Preferably, the pressure generation element comprises a syringe and the pressure generation element can transmit a pressure of 0.1 kilopascals or greater, 1.0 kilopascals or greater, 10 kilopascals or greater, 100 kilopascals or greater, 150 kilopascals or greater, or 200 kilopascals or greater into the tissue. In some embodiments the aperture(s) along the needle barrel have a diameter of about 10 nm-4 mm, 0.01 mm-4 mm, 0.1 mm-4 mm, 1.0 mm-4 mm, 1.5 mm-4 mm, 2.0 mm-4 mm, or 3.0 mm-4 mm The needle barrel can be adapted to transmit an electric current and the device can further comprises an electrode adapted to transmit an electromagnetic field. In some embodiments, the therapeutic agent enters a cell and in others it remains extracellular. In some embodiments, the pressure is transmitted using a fluid medium or a gas medium. In some embodiments, the nucleic acid comprises a sequence from a hepatitis virus such as a hepatitis B antigen (HBV), such as HBcAg, or a hepatitis C virus (HCV) antigen, such as NS3/4A, or a combination thereof such as HBcAg from an HBV virus that infects stork or heron joined to NS3/4A. In other embodiments, the nucleic acid comprises a sequence from a human simian virus antigen. Preferably, the nucleic acid comprises a sequence encoding an antigen capable of generating a proliferative T cell response and in some embodiments, the nucleic acid comprises a sequence from a human immunodeficiency virus.

More embodiments, include hypodermic injection device having a longitudinal axis, the device comprising a connector configured to engage a source of pressurized fluid; and a needle assembly, the needle assembly comprising a stem extending from the connector in a direction substantially parallel to the longitudinal axis of the device, the stem comprising a first lumen that is fluidly coupled with the connector, a first needle barrel extending from the stem in a direction substantially parallel to the longitudinal axis of the device, the first needle barrel comprising a second lumen that is fluidly coupled with the stem and at least one aperture that is fluidly coupled with the second lumen, and a second needle barrel extending from the stem in a direction substantially parallel to the longitudinal axis of the device, the second needle barrel comprising a third lumen that is fluidly coupled with the stem and at least one aperture that is fluidly coupled with the third lumen. In some embodiments, the first needle barrel and the second needle barrel form an injection cavity space there between. In other embodiments, the injection cavity space is configured to receive at least a portion of a subject. In some embodiments, the first needle barrel and second needle barrel each comprise the same number of apertures. In some embodiments, each aperture on the first needle barrel faces an aperture on the second needle barrel. In some embodiments, the first needle barrel and the second needle barrel comprise a pointed distal tip disposed opposite the stem. In some embodiments, the apertures are generally curvilinear. In some embodiments, the apertures are generally polygonal. In some embodiments, the apertures are evenly disposed along a line segment that is substantially parallel to the longitudinal axis of the device. In some embodiments, a third needle barrel extending from the stem in a direction substantially parallel to the longitudinal axis of the device, the third needle barrel comprising a fourth lumen that is fluidly coupled with the stem and at least one aperture that is fluidly coupled with the fourth lumen. In some embodiments, at least one aperture is configured to apply negative pressure to the injection cavity space.

Still more embodiments concern an injection device for delivering a therapeutic agent to subject, the device having a longitudinal axis and comprising a plurality of syringes disposed generally parallel to the longitudinal axis of the device, each syringe comprising a needle with a plurality of apertures disposed along a length of the needle, wherein the apertures face the longitudinal axis of the device. In these embodiments, the at least one syringe comprises a therapeutic agent comprising a gene. In some embodiments, each needle comprises a tip and the tips of the plurality of needles are disposed on a plane that lies substantially normal to the longitudinal axis of the device. Additional embodiments include a hypodermic needle comprising a plurality of apertures distributed along the barrel of said needle, wherein the end of said needle is closed. In some embodiments, said closed end is blunt. In some embodiments, the assembly further comprises a syringe attached to the needle. In some embodiments, said syringe comprises a therapeutic agent, which can be a nucleic acid such as a DNA that encodes a protein. Still more aspects of the invention concern an injection device comprising a plurality of hypodermic needles that comprise a plurality of apertures distributed along the barrel of said needles joined to one or more syringes. Preferably, the end of said needles are closed. In some embodiments, the end of said needles are blunt. In some embodiments, said syringe comprises a therapeutic agent such as a DNA that encodes a protein. In some embodiments, the injection device above comprises a single syringe joined to at least three hypodermic needles. In some embodiments, the at least three hypodermic needles are between about 2 and about 10 mm apart. In some embodiments, the device comprises a single syringe joined to at least four hypodermic needles. In some embodiments, the at least four hypodermic needles are between about 3 and about 6 mm apart. Other embodiments concern a single use hypodermic delivery device comprising a plurality of needles attached to at least one syringe, wherein the needles comprise a plurality of apertures distributed along the barrel of said needles and said at least one syringe comprises a single dose of a therapeutic agent. In some embodiments, the end of said needles are closed. In some embodiments, the end of said needles are blunt. In some embodiments, the therapeutic agent is a nucleic acid. In some embodiments, the nucleic acid is a DNA that encodes a protein. In some embodiments, the device comprises a single syringe joined to at least three hypodermic needles. In some embodiments, the at least three hypodermic needles are between about 2 and about 10 mm apart. In some embodiments, the device comprises a single syringe joined to at least four needles. In some embodiments, the at least four hypodermic needles are between about 3 and about 6 mm apart. Methods of using anyone or more of the aforementioned devices are also embodiments, including a method of delivering a nucleic acid into a cell comprising providing the injection device of anyone of claims 93-101, wherein said device comprises a syringe that comprises a nucleic acid; inserting the needles of said device into a tissue of a subject; and displacing the nucleic acid from the syringe through the needles and into the tissue under conditions that induce the uptake of the nucleic acid by a cell in said tissue. In some embodiments, the nucleic acid is a DNA that encodes a protein. In some embodiments, said DNA encodes a viral antigen. In some embodiments, said viral antigen is an HCV or HBV antigen. Furthermore, in some embodiments a use of a HBcAg or a fragment thereof or a nucleic acid encoding HBcAg or a fragment thereof as an adjuvant. By some approaches, said HBcAg or a fragment thereof or a nucleic acid encoding HBcAg or a fragment thereof is a sequence selected from the group consisting of SEQ. ID NOs. 1-32. A method of enhancing an immune response to an antigen is also an embodiment and said methods are can comprise providing said antigen or a nucleic acid encoding said antigen to a subject in mixture with or shortly after providing said subject with HBcAg or a fragment thereof or a nucleic acid encoding HBcAg or a fragment thereof. In some methods, said HBcAg or a fragment thereof or a nucleic acid encoding HBcAg or a fragment thereof is a sequence selected from the group consisting of SEQ. ID NOs. 1-32. In some methods, the DNA encodes NS3/4A and/or HBcAg (e.g., an HBcAg derived from a virus that infects stork and heron).

### BRIEF DESCRIPTION OF THE DRAWINGS

FIG. 1A illustrates a side view of an embodiment of a hypodermic needle device with two barrels, each barrel having five apertures for delivering a therapeutic agent to an area in between the barrels.

FIG. 1B illustrates a side view of one a embodiment of a hypodermic needle device with four barrels for delivering a therapeutic agent to an area in between the barrels.

FIG. 1C is an image of one embodiment of a hypodermic needle device showing some of the components prior to assembly.

FIG. 1D is an image of one embodiment of a hypodermic needle device showing some of the components, including a hub engaged with a threaded luer adaptor.

FIG. 1E is an image of one embodiment of a hypodermic needle device showing some of the assembled components within the scope of the present application.

FIG. 1F is an image of one embodiment of a hypodermic needle device coupled with a syringe that is within the scope of the present application.

FIG. 1G is an image of a “quadcar” tip with four beveled edges which may be used in the injection devices disclose herein.

FIG. 2A illustrates a side view of an embodiment of a hypodermic needle device with two barrels, each barrel having three apertures for delivering a therapeutic agent to an area in between the barrels.

FIG. 2B illustrates an embodiment of a hypodermic needle with five apertures on each needle that are equally spaced apart.

FIG. 2C illustrates an embodiment of a hypodermic needle with three needles and shows some of the dimension that may be modified according to the teachings of the present application.

FIG. 2D illustrate an embodiment of a hypodermic needle with four needles in a staggered configuration.

FIG. 3 illustrates a side view an embodiment of a hypodermic needle device with two barrels, each barrel having ten apertures for delivering a therapeutic agent to an area in between the barrels.

FIG. 4 illustrates a side view of an embodiment of a hypodermic needle device delivering a therapeutic agent including DNA into a muscle cell of a subject.

FIG. 5A illustrates a side view of an embodiment of a hypodermic needle device with three barrels, each barrel having three apertures for delivering a therapeutic agent to an area in between the barrels.

FIG. 5B is a top view of the hypodermic needle device of FIG. 5A.

FIG. 5C illustrates a side view of an embodiment of a hypodermic needle device with three barrels, each barrel having five apertures for delivering a therapeutic agent to an area in between the barrels.

FIG. 5D illustrates a perspective view of the hypodermic needle device of FIG. 5C delivering a therapeutic agent to the tissue of a subject.

FIG. 6A illustrates a side view of an embodiment of a hypodermic needle device with two barrels, each barrel being disposed at an angle relative to the longitudinal axis of the device.

FIG. 6B illustrates a perspective view of an embodiment of a hypodermic needle device with two barrels and a connector fitting.

FIG. 6C illustrates a top view of the hypodermic needle device of FIG. 6B.

FIG. 7A illustrates a perspective view of an embodiment of a hypodermic needle device with six barrels, each barrel having a plurality of apertures for delivering a therapeutic agent to the tissue of a subject.

FIG. 7B is a top view of the hypodermic needle device of FIG. 7A.

FIG. 8A illustrates a side view of an embodiment of a hypodermic needle device with four barrels, each barrel having a plurality of apertures for delivering a therapeutic agent to the tissue of a subject.

FIG. 8B illustrates a top view of an embodiment of a hypodermic needle device of FIG. 8A.

FIG. 8C illustrates another top view of an embodiment of a hypodermic needle device of FIG. 8A.

FIG. 9 illustrates a top view of an embodiment of a hypodermic needle device including four barrels.

FIG. 10 illustrates a top view of an embodiment of a hypodermic needle device including seven barrels.

FIG. 11 illustrates a top view of an embodiment of a hypodermic needle device including ten barrels.

FIG. 12 illustrates a top view of an embodiment of a hypodermic needle device including three barrels.

FIG. 13 illustrates a top view of an embodiment of a hypodermic needle device including three barrels.

FIG. 14 illustrates a top view of an embodiment of a hypodermic needle device including four barrels.

FIG. 15 illustrates a top view of an embodiment of a hypodermic needle device including four barrels.

FIG. 16 illustrates a top view of an embodiment of a hypodermic needle device including a ring-shaped barrel.

FIG. 17 illustrates a top view of an embodiment of a hypodermic needle device including a ring-shaped barrel.

FIG. 18 illustrates a top view of an embodiment of a hypodermic needle device including a ring-shaped barrel.

FIG. 19 illustrates a cut-away view of an embodiment of a barrel including a single lumen.

FIG. 20 illustrates a cut-away view of an embodiment of a barrel including two lumens.

FIG. 21 is a chart illustrating HCV NS3-specific T cell proliferation as a result of immunization with the HIP injector. Proliferation is measured as radioactivity of cells incubated with antigen divided by the radioactivity of cells incubated with media alone.

FIG. 22A-C are histological evaluations of tissue at the site of injection with a regular 27 gauge needle (FIG. 22A), a small HIP injector (FIG. 22B), and a large HIP injector (FIG. 22C).

FIG. 23A-B is a depiction of a small HIP injector (FIG. 23A) and a large HIP injector (FIG. 23B).

FIG. 24 is a graphical depiction of the radioactivity of cells, as counts per minute, when incubated with various antigens at various concentrations to show radioactive thymidine uptake in a T cell proliferation assay.

FIG. 25A-25I depict various constructs containing the NS3/4A platform and the HBcAg containing NS3 protease cleavage sites.

FIG. 26A-B are examples of the setup for measuring the force requirements when injecting material using one of the injection needle devices disclosed herein.

FIG. 27A-F are top and cross-sectional views of Tests 7-9 showing died water injected into chicken breast.

FIG. 28A-F are top and cross-sectional views of Tests 25-27 showing died water injected into chicken breast.

FIG. 29A-F are top and cross-sectional views of Tests 16-18 showing died water injected into chicken breast.

FIG. 30A-F are top and cross-sectional views of Tests 34-36 showing died water injected into chicken breast.

FIG. 31A-F are top and cross-sectional views of chicken breast having died water injected by hand using a injection needle within the scope of the present application.

FIG. 32A-F are top and cross-section views of chicken breast having died water injected by hand using a single needle.

FIG. 33A-D are perspective and side views of one embodiment of a spring-actuated delivery device for using with the injection needle devices of the present application.

FIG. 34A-D are perspective and side view of one embodiment of a trigger device for using with the injection needle devices of the present application.

FIG. 35A-D are one example of a hub design for the needle devices of the present application.

### DETAILED DESCRIPTION

Aspects of this invention described herein concern devices and methods for the delivery of agents (e.g., nucleic acids) into living tissue. Some embodiments concern an injection device configured to introduce agents, such as nucleic acids, especially DNA, into a target tissue, wherein the molecules are taken up by the cells in a region localized to a site near or proximal to the site of injection.

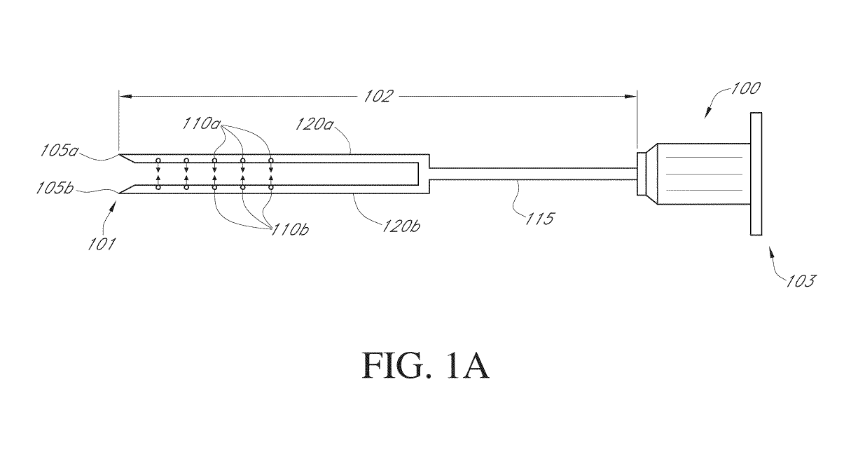

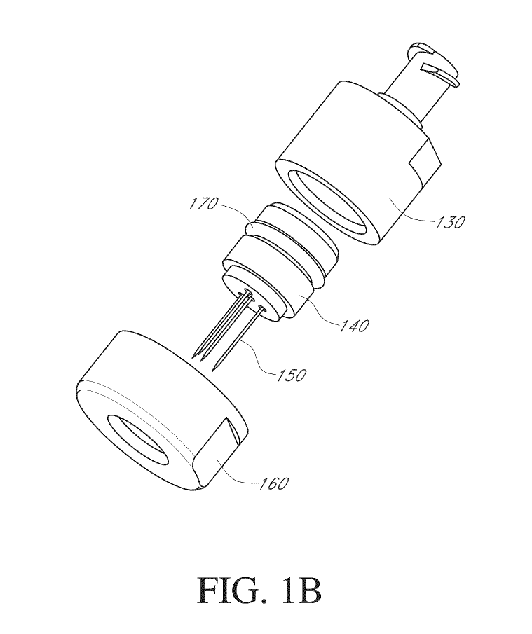

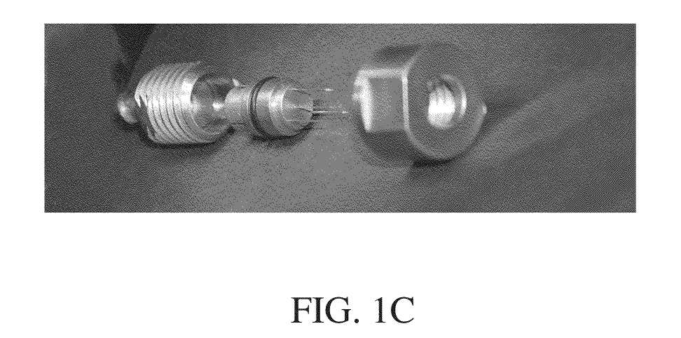

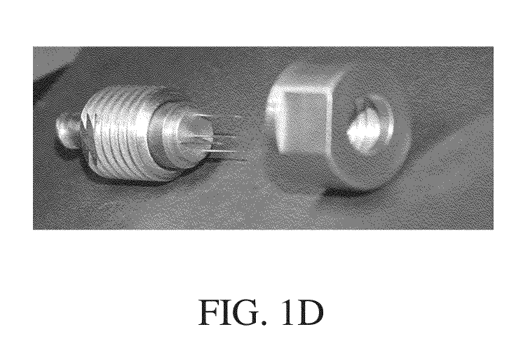

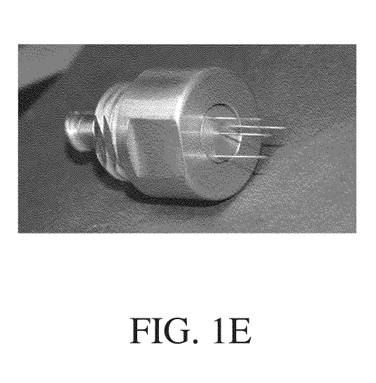

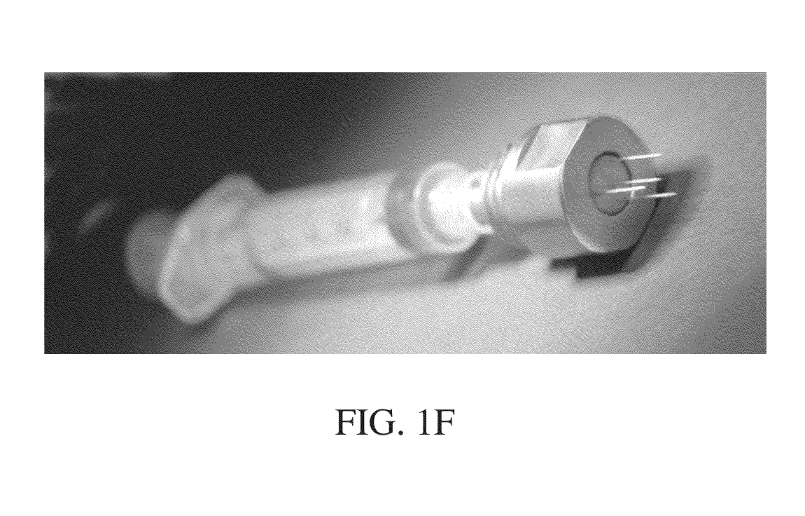

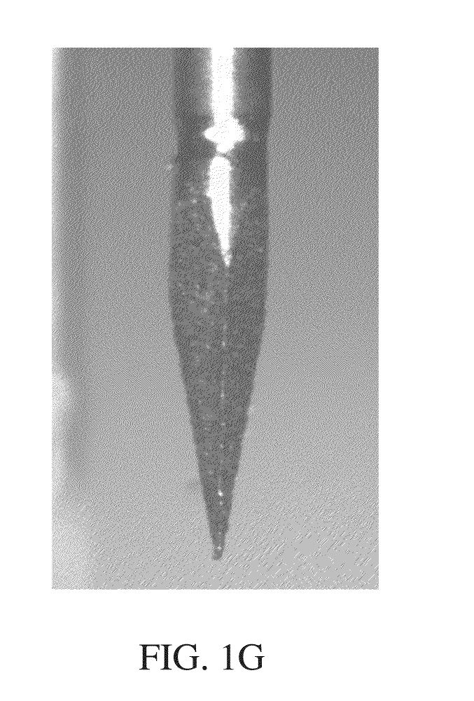

One embodiment of a needle described herein is illustrated in FIG. 1A. The distal tip of the needle can be blunt, beveled, tapered, sharpened, or pointed to permit an operator to pierce the skin of a subject (e.g., a human, domestic animal, such as a cat or dog, or farm animal, such as a horse, cow, pig, or chicken) in order to reach the underlying desired target tissue. For example, the tips 105a, 105b can comprise a regular medical point (e.g., a “lancet point”). Alternatively, the tips 105a, 105b can be blunted. In some embodiments, the distal tip of the needle is closed such that the tip does not establish fluid communication between the lumens of the needle barrel and the distal end of the needle body. In other embodiments, the distal tip is open such that the tip establishes fluid communication between the needle barrel and the distal end of the needle.

In a preferred embodiment, the needle barrel comprises apertures, e.g., 110a, 110b, disposed along a length of the barrel. Each needle barrel can comprise 0 to 100 apertures. In some embodiments, the needle has 1 or 2 apertures along the length of the needle (e.g., a closed ended needle having at least two apertures along the length of the needle). In other embodiments, the needle has a number of apertures that is exactly, less than, or greater than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. The apertures can be located near the distal end of a barrel or anywhere along the length of the barrel. The apertures on each barrel may each be disposed on a plane that is substantially parallel to the longitudinal axis. The apertures can also be disposed along a line segment that is substantially parallel to, and facing, the longitudinal axis of the device. In other embodiments, the apertures may be disposed on one or more planes that are not substantially parallel to the longitudinal axis of the device. Each aperture can face a common point, for example, a point on an axis that is substantially parallel to the longitudinal axis or each aperture can face a different point or direction.

The apertures can vary in size and shape. For example, apertures can be circular, round, generally curvilinear, square, rectangular, triangular, generally polygonal, generally symmetrical, generally asymmetrical, or irregularly shaped. Additionally, the apertures can vary in size and shape within each barrel. For example, in one embodiment, a first aperture on a barrel can be generally curvilinear and have a diameter of about 1 mm and a second aperture on the barrel can have the same shape as the first aperture and have a diameter of about 1.50 mm. In other embodiments, each aperture can have generally the same shape and same size. The apertures can vary in size and shape. For example, apertures can be circular, round, generally curvilinear, square, rectangular, triangular, generally polygonal, generally symmetrical, generally asymmetrical, or irregularly shaped. Additionally, the apertures can vary in size and shape within each barrel. For example, in one embodiment, a first aperture on barrel can be generally curvilinear and have a diameter of about 1 mm and a second aperture on barrel can have the same shape as the first aperture and have a diameter of about 1.50 mm. In other embodiments, each aperture can have generally the same shape and same size.

FIG. 1B illustrates another embodiment of a hypodermic needle within the scope of the present application. Threaded luer adaptor 130 is configured to engage a syringe (not shown) containing a therapeutic material. Hub insert 140 includes plurality of needles 150 at the distal side of hub insert 140. Collar 160 can be configured to engage thread luer adaptor 130 and secure hub insert 140. Gasket 170 may optionally be disposed on hub insert 140 to maintain a sealed channel from a syringe to plurality of needles 150. The needles may optionally include a plurality of apertures (e.g., as depicted in FIG. 1A), as discussed above. FIGS. 1C-E are images of the hypodermic needle illustrated in FIG. 1B and shows an assembly of certain components. FIG. 1F is an image of the assembled hypodermic needle illustrated in FIG. 1B and includes a syringe fluidly coupled to the needles.

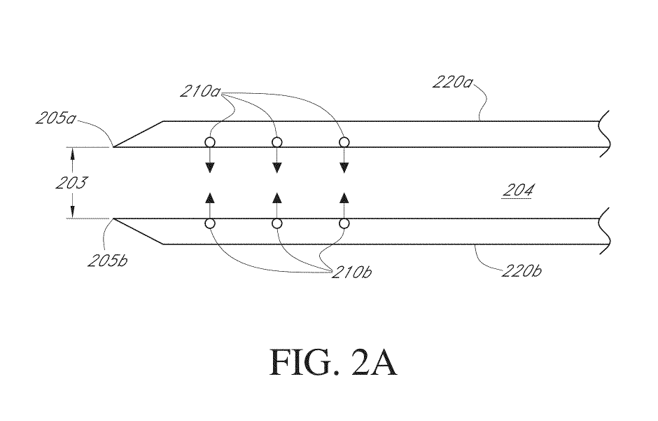

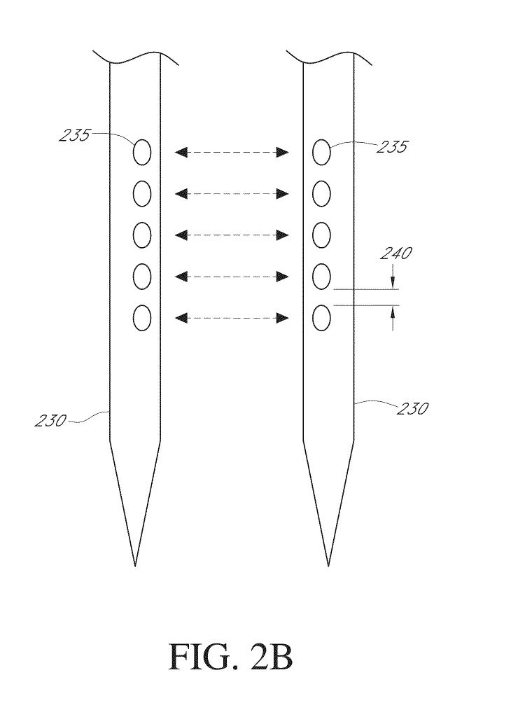

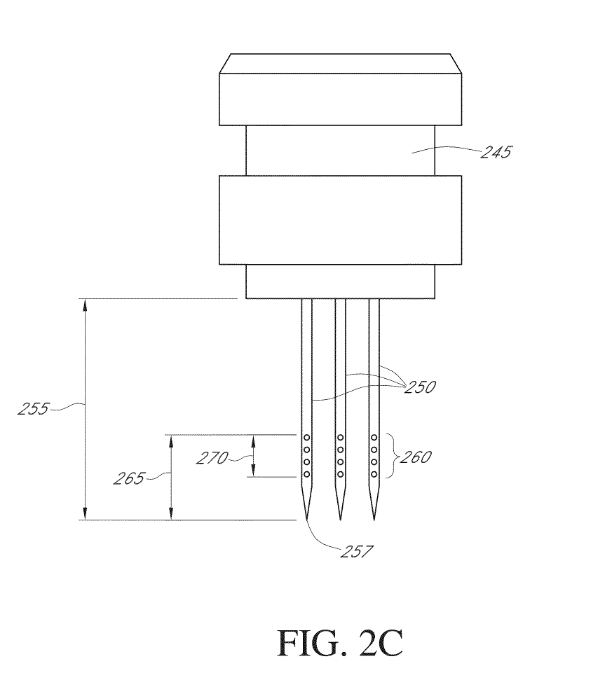

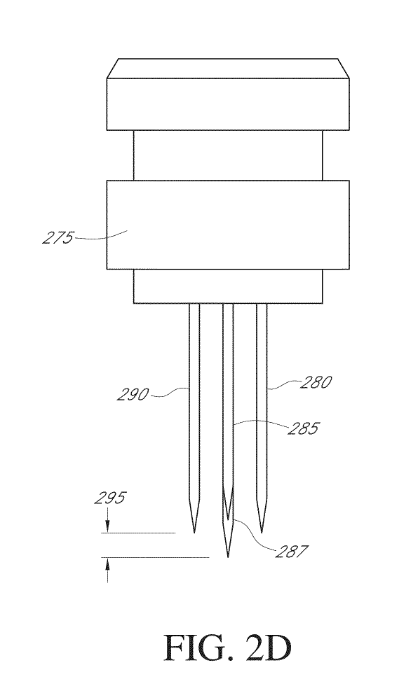

The size, shape, and quantity of apertures can be selected in order to maximize the efficient delivery of injected fluid or genetic material, to create the optimal pressure within the injection cavity space to enhance cell membrane permeability, or to do both. For example, as illustrated in FIG. 2A, in one embodiment, in order to create an injection device for the delivery of a fluid containing a desired agent to targeted tissue, one can select a plurality (e.g., ten) generally curvilinear apertures 210a, 210b with diameters ranging from about 0.01 to about 4.0 mm. In certain embodiments, the width of the apertures 210a, 210b at their widest portion is greater than, less than or equal to about 0.01 mm, 0.02 mm, 0.03 mm, 0.04 mm, 0.05 mm, 0.06 mm, 0.07 mm, 0.08 mm, 0.09 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1.0 mm, 1.05 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm, 2.0 mm, 2.05 mm, 2.10 mm, 2.15 mm, 2.20 mm, 2.25 mm, 2.30 mm, 2.35 mm, 2.40 mm, 2.45 mm, 2.50 mm, 2.55 mm, 2.60 mm, 2.65 mm, 2.70 mm, 2.75 mm, 2.80 mm, 2.85 mm, 2.90 mm, 2.95 mm, 3.0 mm, 3.05 mm, 3.10 mm, 3.15 mm, 3.20 mm, 3.25 mm, 3.30 mm, 3.35 mm, 3.40 mm, 3.45 mm, 3.50 mm, 3.55 mm, 3.60 mm, 3.65 mm, 3.70 mm, 3.75 mm, 3.80 mm, 3.85 mm, 3.90 mm, 3.95 mm, or within a range defined by, and including, any two of these values. In other embodiments, one can select a plurality (e.g., ten) generally curvilinear apertures 210a, 210b with diameters ranging from about 10 nm to about 2.0 mm. In certain embodiments, the width of the apertures 210a, 210b at their widest portion is greater than, equal to, or less than about 0.01 μm, 0.02 μm, 0.03 μm, 0.04 μm, 0.05 μm, 0.06 μm, 0.07 μm, 0.08 μm, 0.09 μm, 0.1 μm, 0.15 μm, 0.2 μm, 0.25 μm, 0.3 μm, 0.35 μm, 0.4 μm, 0.45 μm, 0.5 μm, 0.55 μm, 0.6 μm, 0.65 μm, 0.7 μm, 0.75 μm, 0.8 μm, 0.85 μm, 0.9 μm, 0.95 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 0.1 mm, 0.2 mm, 0.3 mm, 0.4 mm, 0.5 mm, 0.6 mm, 0.7 mm, 0.8 mm, 0.9 mm, 1 mm, 1.05 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm, or 2.0 mm or within a range defined by, and including, any two of these values.

By adjusting the size, shape, and quantity of apertures and taking into account the physical properties of the pressure transmitting medium, the injection device can deliver a local pressure in the range of about 1 to about 200 kilopascals. That is, desirably, the needles described herein are configured to deliver a fluid at a pressure in the range of greater than, less than, equal to, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 kilopascals or any number in between these numbers. An increased local pressure in the tissue contained within the injection cavity space 204 alters the cell membrane permeability characteristics of cells within the tissue and promotes entry of an agent (e.g., DNA) into the cells.

The length of the needle can vary from about 0.5 cm to about 15 cm. In certain embodiments, the needle is, is about, is at least, is at least about, is not more than, is not more than about 0.5, 0.75, 1.0, 1.25, 1.5, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 15.25, 14.5, 14.75, or 15 cm.

Referring again to FIG. 1A, the device includes a proximal end 103, a distal end 101 opposite the proximal end, and a longitudinal axis running from the distal end 101 to the proximal end 103. In some embodiments, the device can contain one or a plurality of needles. In some embodiments, the injection pressure device comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 needles. The device can include a standard connector 100 and a needle body 102 extending from the connector 100. The standard connector 100 and needle body 102 can be disposed on an axis that is substantially parallel to the longitudinal axis. In some embodiments, the standard connector 100 is a luer lock or similar mechanism configured to connect the device to a pressure delivery device (not shown), for example, a syringe or pump.

In some embodiments, a hypodermic injection pressure device contains a therapeutic agent. The device can comprise, for example, a nucleic acid that is formulated for intra muscular delivery. Desirably, DNA encoding an immunogen or a DNA-containing immunogenic composition (e.g., a DNA vaccine) is provided in a device comprising one or more of the needles described herein. However, a wide variety of nucleic acids can be delivered by an embodiment described herein. That is, one or more of the embodiments described herein can comprise one or more of a nucleic acid selected from the group consisting of: mRNA, tRNA, rRNA, cDNA, miRNA (microRNA), siRNA, (small interfering RNA), RNAi (interfering RNA), piRNA (Piwi-interacting RNA), aRNA (Antinsense RNA), snRNA (Small nuclear RNA), snoRNA (Small nucleolar RNA), gRNA (Guide RNA), shRNA (Small hairpin RNA), stRNA (Small Temporal RNA), ta-siRNA (Trans-acting small interfeing RNA), cpDNA, (Chloroplast DNA), gDNA (Genomic DNA), msDNA (Multicopy single-stranded DNA), mtDNA (Mitochondrial DNA), GNA (Glycol nucleic acid), LNA (Locked nucleic acid), PNA (Peptide nucleic acid), TNA (Threose nucleic acid), Morpholino containing nucleic acids, sulfur-containing nucleic acids, 2-O-methyl nucleic acids, and nucleic acids containing one or more modified bases or spacers.

The concentration of the nucleic acid contained in or delivered by a device described herein can vary from about 0.1 ng/ml to about 50 mg/ml. In some aspects, the nucleic acid concentration that is contained in or delivered by a device described herein (e.g., a suitable dose of nucleic acid for delivery by a device described herein) is between about 10 ng/ml to 25 mg/ml. In still other aspects, the nucleic acid concentration is between 100 ng/ml to 10 mg/ml. In some aspects, the nucleic acid concentration contained in or delivered by a device described herein (e.g., a suitable dose of nucleic acid for delivery by a device described herein) is greater than or equal to or less than about 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 950 μg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3.0 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4.0 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, 41 mg/ml, 42 mg/ml, 43 mg/ml, 44 mg/ml, 45 mg/ml, 46 mg/ml, 47 mg/ml, 48 mg/ml, 49 mg/ml, 50 mg/ml, or within a range defined by, and including, any two of these values.

The amount of nucleic acid provided by an injection device described herein can vary from about 1 ng to 10 g. In some aspects, the amount of nucleic acid contained in the hypodermic injection pressure device or provided by the hypodermic injection pressure device is less than greater than or equal to about 1 ng, 5 ng, 10 ng, 20 ng, 30 ng, 40 ng, 50 ng, 60 ng, 70 ng, 80 ng, 90 ng, 100 ng, 150 ng, 200 ng, 250 ng, 300 ng, 350 ng, 400 ng, 500 ng, 600 ng, 700 ng, 800 ng, 900 ng, 1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg 150 μg, 155 μg, 160 μg, 165 μg, 170 μg, 175 μg, 180 μg, 185 μg, 190 μg, 195 μg 200 μg, 205 μg, 210 μg, 215 μg, 220 μg, 225 μg, 230 μg, 235 μg, 240 μg, 245 μg 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg 350 μg, 355 μg, 360 μg, 365 μg, 370 μg, 375 μg, 380 μg, 385 μg, 390 μg, 395 μg 400 μg, 405 μg, 410 μg, 415 μg, 420 μg, 425 μg, 430 μg, 435 μg, 440 μg, 445 μg 450 μg, 455 μg, 460 μg, 465 μg, 470 μg, 475 μg, 480 μg, 485 μg, 490 μg, 495 μg 500 μg, 505 μg, 510 μg, 515 μg, 520 μg, 525 μg, 530 μg, 535 μg, 540 μg, 545 μg 550 μg, 555 μg, 560 μg, 565 μg, 570 μg, 575 μg, 580 μg, 585 μg, 590 μg, 595 μg 600 μg, 605 μg, 610 μg, 615 μg, 620 μg, 625 μg, 630 μg, 635 μg, 640 μg, 645 μg 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg 850 μg, 855 μg, 860 μg, 865 μg, 870 μg, 875 μg, 880 μg, 885 μg, 890 μg, 895 μg 900 μg, 905 μg, 910 μg, 915 μg, 920 μg, 925 μg, 930 μg, 935 μg, 940 μg, 945 μg 950 μg, 955 μg, 960 μg, 965 μg, 970 μg, 975 μg, 980 μg, 985 μg, 990 μg, 995 μg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, 10.0 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 2 g, 3 g, 4 g, 5 g, 6 g, 7 g, 8 g, 9 g, 10 g or within a range defined by, and including, any two of these values.

In some embodiments, the device can be configured to be a one-time disposable device, wherein the therapeutic agent is contained within the device and no additional connection is required. The needle body 102 can include one or more needle delivery barrels or needle barrels 120a, 120b that extend from a stem or cannula 115. The stem 115 can include a central lumen or channel Each needle barrel 120a, 120b also includes at least one lumen that is fluidly connected to the stem 115 and standard connector 100. In the illustrated embodiment, the needle body 102 includes two needle delivery barrels 120a, 120b with each needle barrel 120 including a distal tip 105a, 105b. The lengths of the needle barrels 120a, 120b can vary. In some embodiments, the needle barrels 120a, 120b are each about the same length and in other embodiments, the needle barrels are different lengths. The needle barrels 120a, 120b can range from about 2 mm to about 100 mm. The gauges of the needles barrels 120 can vary from device to device or from barrel 120 to barrel 120 on a single device, as well.

Although the tips 105a, 105b are shown with the beveling angling towards the longitudinal axis of the device, the bevels may be angled in the opposite direction (see FIG. 2A), or different directions (see FIG. 4), in order to spread tissue and deliver at least some targeted tissue through an area disposed between the needle barrels 120a, 120b and into an injection cavity space disposed therebetween. In some embodiments, each tip can include multiple beveled edges, such two, three, four, five, six, or more beveled edges. This can result in a tip having generally a rotational symmetry about its axis and may provide for uniform insertion of each needle. FIG. 1G is an image of a “quadcar” tip having four beveled edges which may used on one or more needles in the injection devices disclose herein. In some embodiments, at least one beveled edge on the needle tip faces generally the same direction as one or more apertures on the same needle. In some embodiments, none of the beveled edges on the needle tip face in generally the same direction as any of the apertures on the same needle. In some embodiments, the opening created by the space between the needle barrels 120a, 120b at the distal end of the device is sufficiently large in size to enable the needle barrels 120a, 120b to surround one or more cells.

The needle barrels 120a, 120b can each comprise apertures 110a, 110b disposed along a length of the barrels. In some embodiments, each needle barrel 120a, 120b comprises at least one aperture 110a, 110b. In other embodiments, at least one needle barrel 120a, 120b does not comprise an aperture 110a, 110b. In some embodiments, the size and shape of each aperture 110a, 110b can vary from barrel to barrel. In some embodiments, the length of the needle can vary from barrel to barrel.

Referring again to FIG. 2A, the injection device includes two needle barrels 220a, 220b each including three apertures 210a, 210b and a pointed distal tip 205a, 205b. The distal tips 205a, 205b are separated from one another by a distance to form an opening 203. Moving in the proximal direction from the distal tips 205a, 205b the opening 203 forms an injection cavity space 204 formed between the needle barrels 220a, 220b. In some embodiments, the opening 203 created by the space between needle barrels 220a, 220b between the tips 205a, 205b is sufficiently large in size to enable the needle barrels 220a, 220b to surround one more or cells in the injection cavity space 204.

Delivering an agent at a suitable local pressure within the cavity space may be important for effective and safe treatment. For example, applying too much pressure may result in undesirable damage to the cell, while applying too little pressure may not yield a sufficient permeability shift so as to allow for uptake of the agent. The laws of fluid dynamics and associated equations can be used to generate a profile of acceptable pressures in the injection cavity space 204. For example, the needle barrel 120a, 120b geometry and the fluid characteristics of the agent, for example, viscosity and density, will affect the local pressure in the injection cavity space 204. In some embodiments, the size and shape of the apertures 210a, 210b, the fluid and delivered agent, as well as, the driving pressure are selected by the user to produce a desired local pressure in the injection cavity space 204. The Darcy-Weisbach equation, for example, may be used to define the pressure drop with regards to the velocity of flow, the viscosity of the fluid, and the ratio of the diameter of the barrel lumen to the pipe length. The equation is useful, among other things, in determining the appropriate aperture 210a, 210b size when using different carrier medium fluids (e.g. phosphate buffered saline, glycerin, ethanol, deionized water, filtered water, various oils, emulsions, etc.), as each type of fluid has its own viscosity properties. Standard computational fluid dynamics software can be utilized in determining the optimal physical parameters of the needle barrels and apertures to achieve a desired pressure drop. However, the invention is not limited to the use of fluid for the creation of the pressure drop, and can utilize other types of pressure transmitting mediums. For instance, in some embodiments, air or other gas, such as CO₂ or N₂, may be used to transmit pressure onto tissue.

FIG. 2B illustrates another example of needles having a plurality of apertures. Needles 230 each include five apertures 235 having spacing 240 between each aperture. The spacing between the apertures may, in some embodiments, be the same for all the apertures in the needles, or they can be different. The spacing may, for example, be about, at least, at least about, not more than, not more than about 0.01 mm, 0.05 mm, 0.1 mm, 0.15 mm, 0.2 mm, 0.25 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 1 cm, 2 cm or 3 cm. Apertures 235 are configured such that each aperture faces a second aperture on a different needle. This can result in opposing fluid flow of the therapeutic material between apertures that face each other. In some embodiments, all of the apertures are configured to face (or oppose) another aperture on a different needle (e.g., as depicted in FIG. 2B). In some embodiments, at least 2, 4, 6, 8, 10, 16, 20, 30, 40, 50, or 60 of the apertures are configured to face (or oppose) another aperture on a different needle.

FIG. 2C illustrates another embodiment of the needle device and various dimensions that may be modified according the present application. Hub 245 includes three needles 250 fluidly coupled to the distal end of hub 245. Needles 250 each have a needle length 255 from the distal end of hub 245 to needle point 257. As discussed further in the application, needle length 255 may vary depending upon the target tissue for delivering a therapeutic material. Distance 265 between needle point 257 and the aperture on the needle furthest from needle point 257 can also be varied. For example, distance 265 may be between 0.1 mm and 5 cm, such as about 1 mm, 2 mm, 3 mm, 4 mm, 5 mm, 6 mm, 7 mm, 8 mm, 9 mm, 1 cm, 2 cm, 3 cm, 4 cm or more. Similarly, distance 270 between the aperture closest to needle point 257 and the aperture furthest from needle point 257 may also vary. In some embodiments, distance 257 may be between 0.5 mm and 10 cm, such as 1 mm, 2 mm, 3 mm, 4 mm, 5 mm, 6, mm, 7 mm, 8 mm, 9 mm, 1 cm, 2 cm, 3 cm, 4 cm, 5 cm, 6 cm, 7 cm, 8 cm, 9 cm, or more.

FIG. 2D illustrates another embodiment of an injection device having needles in a staggered configuration. Hub 275 includes four needles 280, 285, 287, 290 fluidly coupled to the distal end of hub 245. Needle 287 is longer than needle 290 by distance 295. Meanwhile, needle 280 is longer than needles 285, 290 but shorter than needle 287. Numerous other variations of the staggered arrangement may also be used. In some embodiments, the injection device includes a plurality of needles, where at least one or more needles have a first length and one or more needles have second length that is longer than the first length. In some embodiments, the injection device includes a plurality of needles, where each needle has a different length (e.g., as depicted in FIG. 2D). The difference in length between the needles may, for example, be at least 0.1 mm, 0.15 mm, 0.2 mm, 0.25 mm, 0.3 mm, 0.4 mm, 0.5 mm, 0.6 mm, 0.7 mm, 0.8 mm, 0.9 mm, 1 mm, 2 mm, 3 mm, 4 mm, or 5 mm The difference in length between the needles may, for example, be no more than 5 cm, 2 cm, 1 cm, 5 mm, 4 mm, 3 mm, 2 mm, or 1 mm.

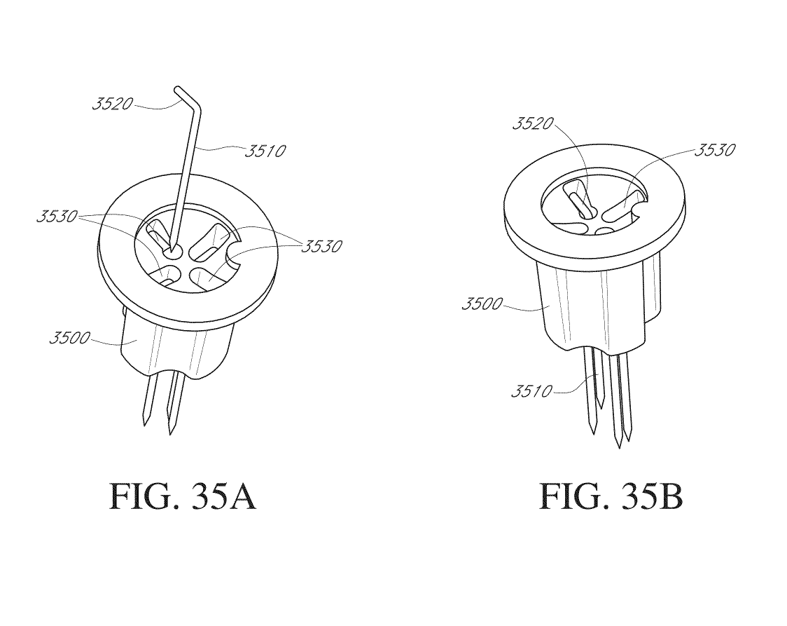

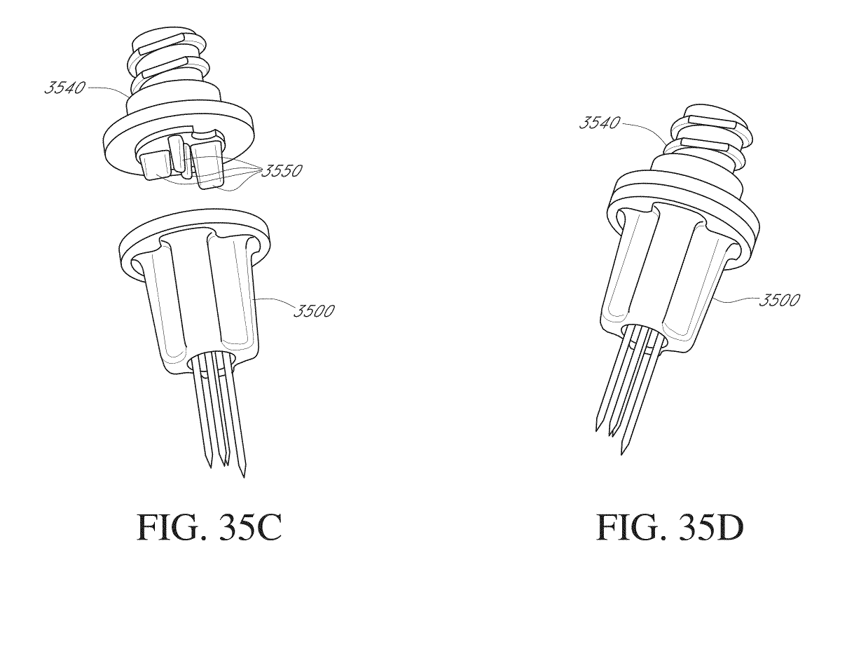

FIG. 35A shows one embodiment of a hub design that may be included within the needle devices. Bottom-hub component 3500 is configured to receive a plurality of needles, each needle having needle barrel 3510 and hub-engaging member 3520 disposed at one end of the needle. Bottom-hub component 3500 includes apertures 3530 that receive the needle barrel 3510 and engage hub-engaging member 3520 to maintain the needle within the hub. FIG. 35B shows the needles after being inserted within apertures 3530. The depth of apertures 3530 may vary so that the needles are staggered relative to each other (e.g., as depicted in FIG. 2D). FIG. 35C shows top-hub component 3540 having aperture-engaging members 3550 that are configured to engage apertures 3530 when top-hub component 3540 is disposed on bottom-hub component 3500. Aperture-engaging members 3550 can secure the hub-engaging member 3520 within the hub. FIG. 35D shows the hub having bottom-hub component 3500 and top-hub component 3540 secured together by, for example, welding the two components together.

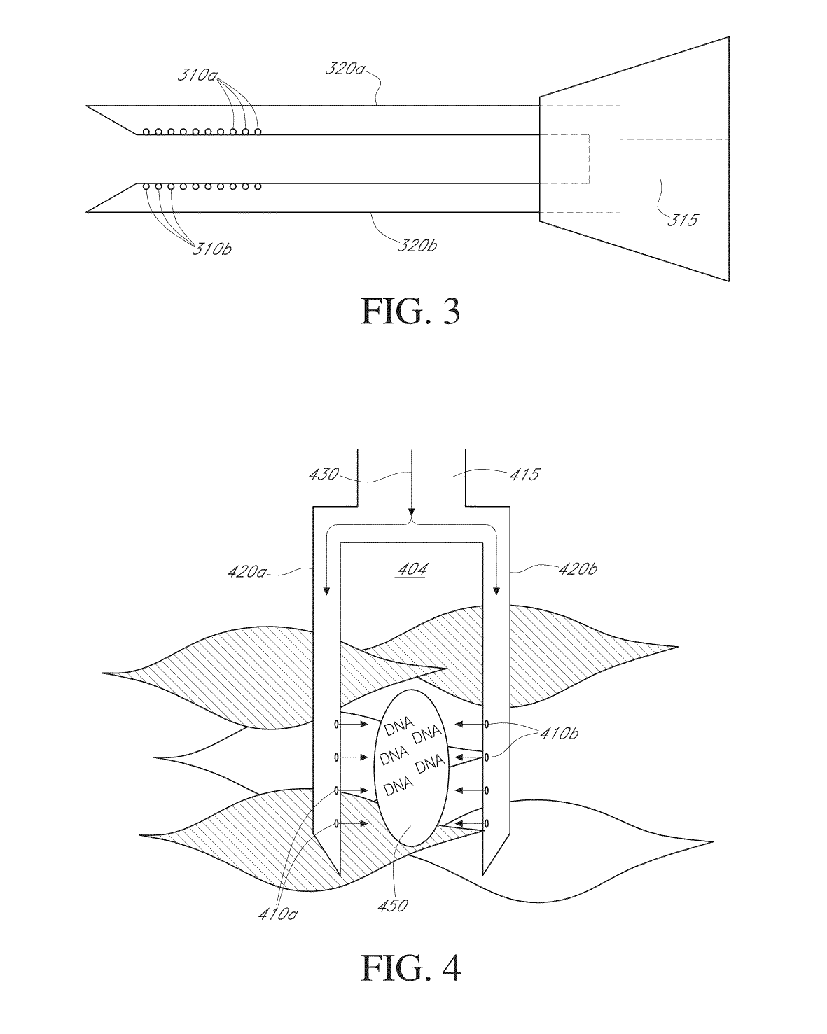

Turning now to FIG. 3, another embodiment of an injection device including two needle barrels 320a, 320b is illustrated. The needle barrels 320a, 320b include lumens that are in fluid communication with a central lumen 315. A pressurized therapeutic agent can be directed through the central lumen 315 to the needle barrels 320a, 320b and can exit the needle barrels 320a, 320b via apertures 310a, 310b. In this embodiment, the needle barrels 320a, 320b each comprise ten curvilinear apertures evenly distributed along a distal length of the barrels. The apertures 310a, 310b are configured to direct the pressurized agent towards the longitudinal axis of the device and thus, the apertures 310a on needle barrel 320a face the apertures 310b on needle barrel 320b. In one embodiment, the apertures can be disposed proximally from the tips of the barrels 320a, 320b between about 1 and about 3 mm towards the proximal ends of the barrels.

FIG. 4 illustrates the injection of a fluid therapeutic agent 430 into a cell 450. The therapeutic agent 430 can carry a gene, a nucleic acid, protein, or other large molecule into part of a cell 450 or into multiple cells, as described above. In the illustrated example, the injection device has been introduced into the muscle tissue such that the injection cavity space 404 surrounds at least part of one muscle cell 450. A high pressure source of fluid (not shown) is directed into the central lumen 415 of the device and through the lumens of each of the needles barrels 420a, 420b before it is expelled through the apertures 410a, 410b into the injection cavity space 404. The high pressure that exists at each aperture 410a, 410b results from pressure applied to the fluid as it is expelled into the tissue located in the injection cavity space 404. The resulting increase in local pressure alters the permeability properties of the membrane in order to enhance uptake of the injected element. The resulting permeability change allows pharmaceutical drugs, nucleic acids and other compounds to gain access to the interior of the cell.

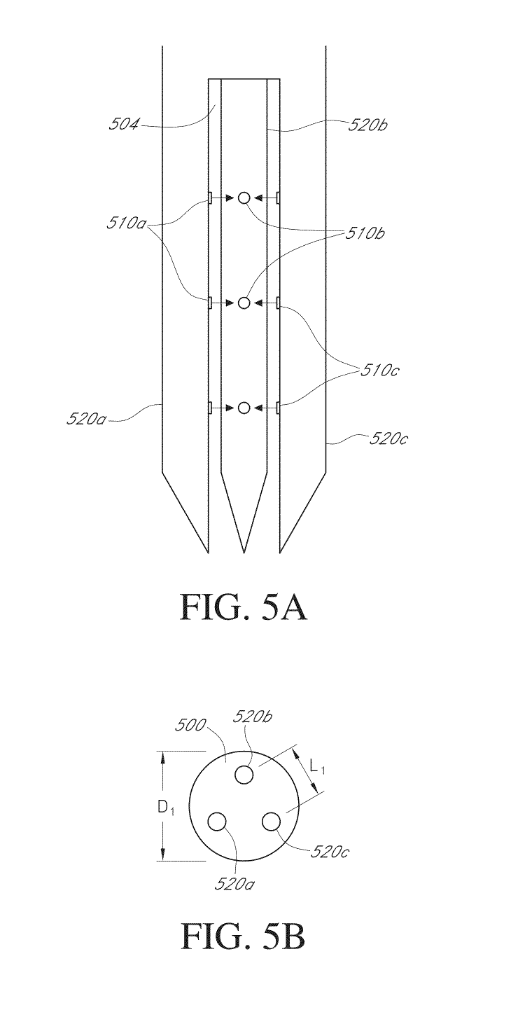

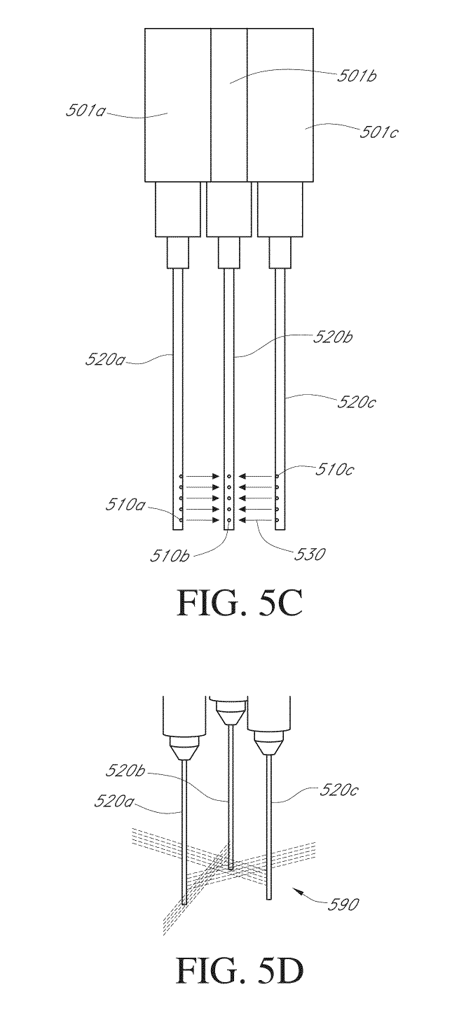

As mentioned above, the number of needle barrels can vary depending on the intended application for the injection device, the manufacturing process used to create the injection device, the amount of local pressure desired, and/or other factors. In some embodiments, the number of barrels can be equal or greater than 1, 2, 3, 4, 5, 6, 7, 8, 8, 10, or more. For example, in the embodiment illustrated in FIG. 5A, three needle barrels 520a, 520b, 520c extend longitudinally to form an injection cavity space 504 therebetween. In the illustrated embodiment, each needle barrel 520a, 520b, 520c includes three apertures 510a, 510b, 510c evenly disposed along an inner facing contour of the barrels.

FIG. 5B illustrates a top view of the injection device shown in FIG. 5A. The needle barrels 520a, 520b, 520c can each be disposed around the center of the connector or central lumen housing 500. The needle barrels can form a triangle, for example, an equilateral triangle. The diameter D₁ of the connector 500 can vary as can the length L₁ between the needle barrels 520. In one embodiment, the diameter D₁ of the connector 500 ranges from about 3 to about 25 mm and the length L₁ between the needle barrels ranges from about 1 to about 8 mm, or more.

FIG. 5C illustrates a side view of an embodiment of an injection device including three separate syringes 501a, 501b, 501c. The syringes can be configured to contain similar or different volumes of a therapeutic agent for delivery to a patient. In one embodiment, each syringe is configured to contain 1 mL of a therapeutic agent. Each syringe 501a, 501b, 501c includes a needle barrel 520a, 520b, 520c extending longitudinally therefrom. Each needle barrel 520 includes a plurality of apertures 510a, 510b, 510c facing the longitudinal axis of the device. The number of apertures 510a, 510b, 510c on each needle barrel 520a, 520b, 520c can range from one to twenty. In one embodiment, the apertures 510 on a barrel 520 are evenly distributed with one aperture disposed about over 0.2 mm The volume range per length of needle barrel 520 can vary depending on the distance between apertures 510. In one embodiment, each millimeter of length of needle barrel 520 corresponds to 75 μl of therapeutic agent. The three syringes 501 can be arranged in an equilateral triangle shape centered around the longitudinal axis of the device with each needle barrel 520 being about equal distance from each of the other two needle barrels.

The distance between the needle barrels 520 can vary depending on the number of apertures 510. In one embodiment, each needle barrel 520 comprises ten apertures 510 and the needles are disposed about 3.0 mm apart from one another. In another embodiment, each needle barrel 520 comprises 8 apertures 510 and the needles are disposed about 2.2 mm apart from one another. In another exemplary embodiment, each needle barrel 520 comprises six apertures and the needles are disposed about 1.5 mm apart from one another. In yet another embodiment, each needle barrel 520 comprises about 4 apertures 510 and the needles are disposed about 1.0 mm apart from one another.

FIG. 5D illustrates a perspective view of the injection device of FIG. 5C delivering a therapeutic agent to a subject 590.

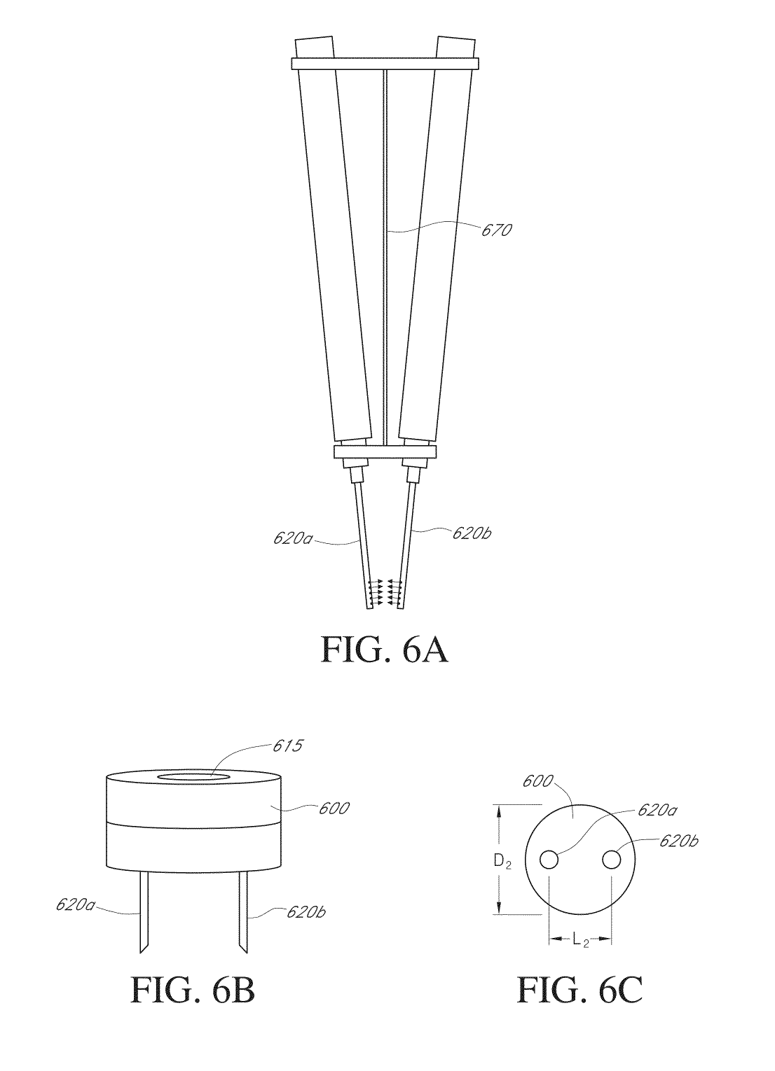

Turning now to FIG. 6A, another embodiment of a multiple syringe injection device is illustrated. The injection device in FIG. 6A includes two syringes 620a, 620b each disposed at an angle relative to the longitudinal axis of the device. A support 670 holds the syringes 620 in position relative to one another and is generally aligned with the longitudinal axis of the device.

FIG. 6B illustrates a perspective view of another embodiment of an injection device including two needle barrels 620a, 620b that are fluidly connected to a common lumen 615 that is housed within a housing or connector 600. In this embodiment, the needle barrels 620a, 620b are generally parallel to one another and distribute a therapeutic agent to a subject that is directed to the barrels by the common lumen 615. FIG. 6C illustrates a top view of the connector 600 and needles barrels 620a, 620b of FIG. 6B. The needles barrels 620a, 620b can be separated one another be a length L₂ and the connector 600 can have a diameter or width D₂. The diameter D₂ of the connector 600 can vary as can the length L₂ between the needle barrels 620. In one embodiment, the diameter D₂ of the connector 600 ranges from about 3 to about 25 mm and the length L₂ between the needle barrels ranges from about 1 to about 6 mm.

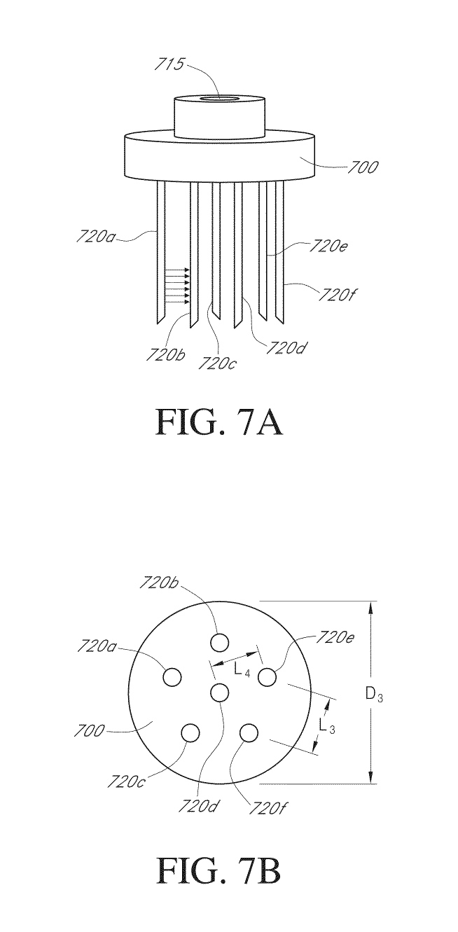

FIG. 7A illustrates another embodiment of an injection device including six needle barrels 720 extending generally parallel to one another from a connector 700. The connector 700 houses a common lumen 715 that distributes a pressurized therapeutic agent to the needle barrels 720. FIG. 7B illustrates a top view of the injection device of FIG. 7A. As shown in FIG. 7B, five of the needles barrels 720 can form a pentagram or five-sided polygon centered around the center of the connector 700. Each of these five needle barrels 720 can be separated from a left and right needle barrel 720 by a length L₃. The sixth needle barrel 720 can be disposed in the center of the five-sided polygon and separated from the other five needle barrels by a length L₄. The connector 700 can also have a diameter of maximum width D₁. In some embodiments, the diameter D₁ can be between about 3 and about 25 mm. The lengths L₄ and L₃ can be equal to one another or different. In some embodiments, length L₄ ranges from about 1 to about 6 mm and length L₃ ranges from about 1 to about 6 mm.

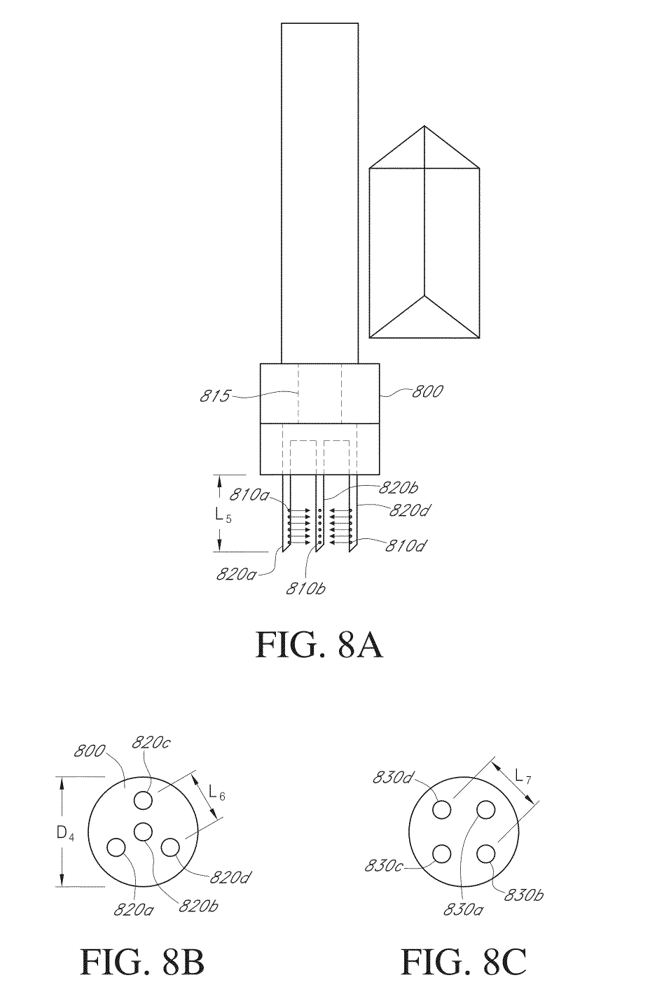

FIG. 8A illustrates another embodiment of an injection device including four needle barrels 820 that fluidly connect with a common lumen 815 housed within a connector 800. Each needle barrel 820 can include any number of inner facing apertures 810, for example, six or ten. In some embodiments, a needle barrel 820 is disposed along the longitudinal axis of the device and includes no apertures 810 or includes apertures 810 that face away from the center or longitudinal axis of the device. For example, needle barrel 820b can include three zones containing apertures, where each zone includes apertures (e.g., six apertures) that face one needle selected from needle 820a, needle 820c or needle 820d. The needle barrels 820 can extend from the connector 800 for a length L₅ between about 3 and about 100 mm FIG. 8B illustrates a top view of the injection device of FIG. 8A including the connector 800 and the needle barrels 820. Three of the needle barrels 820 can be disposed in a triangle, for example, an equilateral triangle, centered around the longitudinal axis of the device and sharing a common center with the connector 800. These needle barrels 820 can be separated from one another by a length L₆. This length L₆ can vary between about 2 and about 12 mm. For example, L₆ can be about 3 mm or about 6 mm The connector 800 can have a diameter or maximum width D₄ dimension ranging from about 3 to about 20 mm.

FIG. 8C illustrates a top view of another embodiment of the injection device. Needles 830 form points of a square (or any other quadrilateral, such as a trapezoid, isosceles trapezoid, parallelogram, kite, rhombus, or rectangle) having a length L₇ between needle 830d and needle 830b. This length L₇ can vary between about 2 and about 12 mm, such as 3 mm or 6 mm. In some embodiments, each needle may be configured with a first zone of apertures that face a first adjacent needle. For example, needle 830b may include a first zone of apertures that face needle 830a. In some embodiments, each needle may be configured with a second zone of apertures that oppose a second adjacent needle. For example, needle 830b may include a first zone of apertures that face needle 830a and a second zone of apertures that face needle 830c. In some embodiments, each needle may be configured with a third zone of apertures that oppose a third adjacent needle. For example, needle 830b may include: a first zone of apertures that face needle 830a, a second zone of apertures that face needle 830c, and a third zone of apertures that face needle 830d. In some embodiments, each needle is configured with the same number of zones. In some embodiments, each zone includes the same number of apertures. Needles 830 may optionally be configured to form a diamond-shape, such as a parallelogram or rhombus.

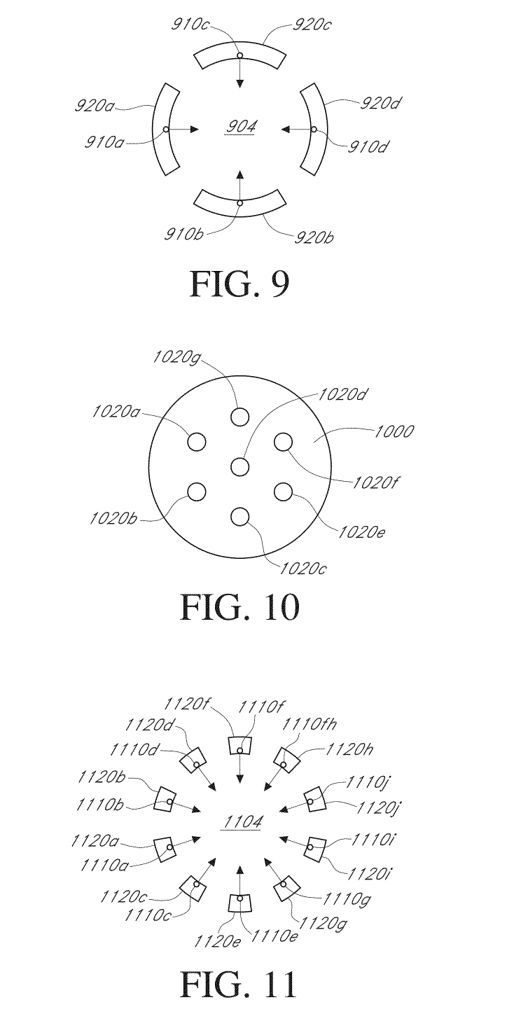

FIGS. 9-15 illustrate top views of various other embodiments of injection devices. Each of these injection devices includes a plurality of needle barrels and can include apertures disposed on the needle barrels. The apertures can be configured to deliver a pressurized therapeutic agent to a subject and/or apply a negative pressure to a subject.

FIG. 9 illustrates an embodiment of an injection device having four needle barrels 920 with each needle barrel comprising at least one inward or center facing aperture 910 configured to deliver a pressurized therapeutic material into an injection space 904.

FIG. 10 illustrates an embodiment of an injection device having seven needle barrels. Six of the needle barrels 1020 from a hexagon with the seventh needle barrel disposed near the center of the hexagon.

FIG. 11 illustrates an embodiment of an injection device having ten needle barrels 1120 with each needle barrel comprising at least one inward or center facing aperture 1110 configured to deliver a pressurized therapeutic material into an injection space 1104.

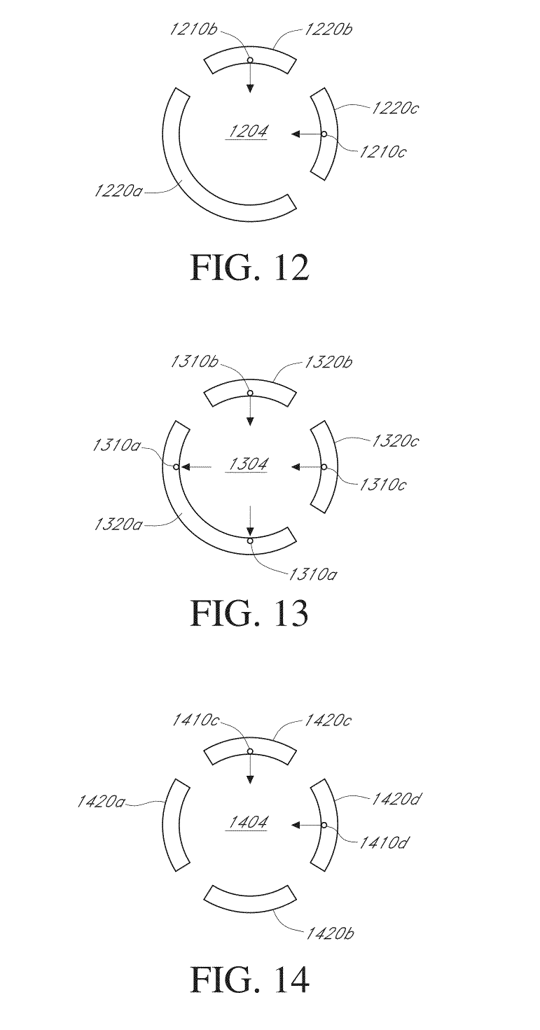

FIG. 12 illustrates an embodiment of an injection device having three needle barrels 1220 with two of the three needle barrels comprising at least one inward or center facing aperture 1210 configured to deliver a pressurized therapeutic material into an injection space 1204. The third needle barrel 1220 does not comprise any apertures configured to deliver pressurized fluid to the injection space 1204.

FIG. 13 illustrates an embodiment of an injection device having three needle barrels 1320 with two of the three needle barrels comprising at least one inward or center facing aperture 1310 configured to deliver a pressurized therapeutic material into an injection space 1304. The third needle barrel 1320 comprises at least two inward or center facing apertures 1310 configured to apply a negative pressure to the injection space 1304.

FIG. 14 illustrates an embodiment of an injection device having four needle barrels 1420 with two of the four needle barrels comprising at least one inward or center facing aperture 1410 configured to deliver a pressurized therapeutic material to an injection space 1404. The third and fourth needle barrels 1420 do not comprise any apertures configured to deliver pressurized fluid into the injection space 1404.

FIGS. 12-14 illustrate embodiments of injection devices where a pressurized therapeutic agent is delivered asymmetrically about an injection cavity space. This may be desirable in some circumstances, for instance, to deliver more focused positive pressure on only a portion or region of the tissue, rather than on all sides.

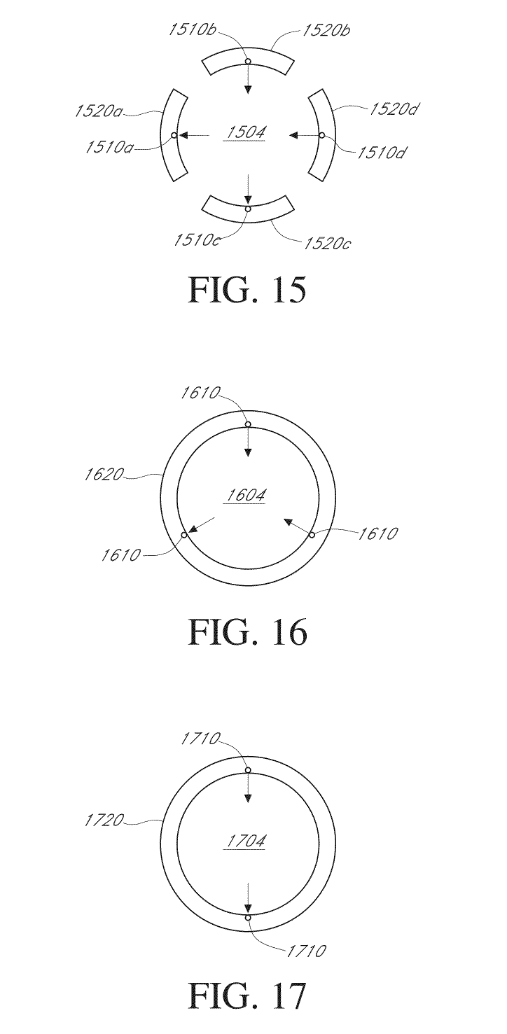

FIG. 15 illustrates an embodiment of an injection device having four needle barrels 1520 with two of the four needle barrels comprising at least one inward or center facing aperture 1510 configured to deliver a pressurized therapeutic material into an injection space 1504. The third and fourth needle barrels 1520 comprise apertures configured to apply a negative pressure to the injection space 1504.

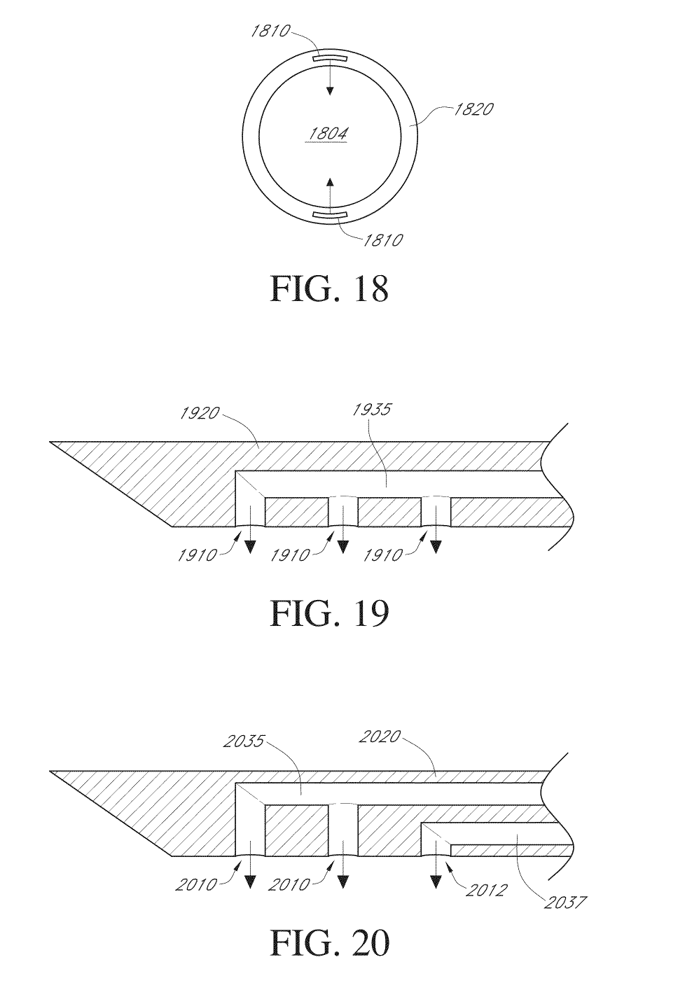

As mentioned above, the shape of each needle barrel can vary. FIGS. 16-18 illustrate embodiments of ring shaped needle barrels that include inward or center facing apertures. FIG. 16 illustrates a needle barrel 1620 that is ring shaped and includes three inward or center facing apertures 1610. Two of the three apertures 1610 are configured to deliver a pressurized therapeutic material into an injection space 1604 and the third aperture 1610 is configured to apply a negative pressure to the injection space. The apertures 1610 can form a triangle, for example, an equilateral triangle. FIG. 17 illustrates a needle barrel 1720 that is ring shaped and includes two inward or center facing apertures 1710 that face one another. One of the two apertures 1710 is configured to deliver a pressurized therapeutic material into an injection space 1704 and the other aperture 1710 is configured to apply a negative pressure to the injection space. FIG. 18 illustrates a needle barrel 1820 that is ring shaped and includes two inward or center facing apertures 1810 that face one another. Both of the apertures 1820 are configured to deliver a pressurized therapeutic material into an injection space 1804. The apertures 1810 can comprise any suitable shape, for example, a slit or generally polygonal shape.

FIGS. 13 and 15-17 illustrate embodiments wherein an injection device is configured to apply negative pressure via one or more apertures to an injection cavity space. Negative or counter-pressure can be used to deliver an optimal amount of pressure onto a cell membrane. In these embodiments, negative pressure is represented by arrows directed toward one or more of the needle barrels. Negative pressure can be applied by connecting certain apertures to a different lumen than other apertures.

In some embodiments, a needle barrel can comprise one lumen that is fluidly connected to a plurality of apertures or more than one lumen. FIG. 19 illustrates a needle barrel 1920 that includes a single lumen 1935 and three apertures 1910 that are each fluidly connected with the single lumen 1935. The lumen 1935 is used for both the transmission of pressure and the delivery of the therapeutic agent. FIG. 20 shows an embodiment wherein a needle barrel 2020 includes a first lumen 2035 that is fluidly connected with two apertures 2010. The needle barrel 2035 also includes a second lumen 2037 that is fluidly connected with a third aperture 2012. This embodiment can be employed, for example, if it becomes desirable to use a first lumen for the delivery of a pressurized therapeutic agent and a second lumen for the delivery of another fluid and/or the application of negative pressure, or vice-versa.

The needle barrels and embodiments described herein may be used in conjunction with other known methods and systems for enhancing gene delivery such as the electroporation system described in U.S. Pat. No. 6,610,044 to Mathiesen, which is hereby incorporated by reference in its entirety. Accordingly, some embodiments of the present invention utilize control circuitry to generate an electric current or an electromagnetic field to alter cell permeabilities. In some embodiments, it may be desired to utilize one or more of the needle barrels themselves to conduct or transmit the generated current or field into the tissue. Indeed, the needle barrels may be used in conjunction with any number of known alternative microporation methods using optionally one or more of sonic, electromagnetic, mechanical and thermal energy or a chemical enhancer, such as that disclosed in U.S. Pat. No. 6,527,716 to Eppstein, which is included by its entirety herein.

Embodiments disclosed herein are not limited to any particular manufacturing process to create the barrels or apertures disclosed. The needle barrels can be manufactured using any of the standard needle manufacturing techniques including, by way of example only, die-casting, injection molding, blow molding, machine tooling, laser fabrication and others. Similarly, the material for the needle can be chosen from any number of well-known needle materials such as stainless steel, carbon steel, and various metal alloys. The apertures on the barrels can be created as a part of the barrel manufacturing process, or can be added later by drilling or laser etching. These various manufacturing methods are all well-known in the art.

Aspects of the present invention also relate generally to methods of transmembrane delivery of drugs, nucleic acids, or other bioactive molecules and compounds using the HIP needle described above. The active ingredients (e.g. DNA, RNA, nucleic acids, protein, or compounds) can be formulated in a number of solutions for delivery through the needles described herein. In some embodiments, the active ingredients (e.g. DNA, RNA, nucleic acids, protein, or compounds) may be mixed in with a carrier solution such water, a buffer, saline, an oil emulsion, oil, or glycerin. The liquid can then be passed through a needle as described herein. In some embodiments the active ingredients (e.g. DNA, RNA, nucleic acids, protein, or compounds) can be attached to a support (e.g. a nanoparticle, protein, sugar, or pellet) and mixed with one or more of the aforementioned carrier solutions (e.g. water, a buffer, saline, an oil emulsion, oil, or glycerin) and the support bound agent is passed through the needles described herein. It will be understood that there exists a variety of carrier mediums and supports, and using carrier mediums or supports not specifically mentioned herein will not depart from the spirit of the invention. For instance, the carrier medium may be a cationic oil.

The nucleic acid contemplated for use with the injection device described herein can be nucleic acids from human, non-human primates, mice, bacteria, viruses, mold, protozoa, bird, reptiles, birds—such as stork, and heron, mice, hamsters, rats, rabbits, guinea pigs, woodchucks, pigs, micro-pigs, goats, dogs, cats, humans and non-human primates, e.g., baboons, monkeys, and chimpanzees, as mentioned above. In certain embodiments, the injection device described herein can be used for the delivery of nucleic acids encoding proteins found in the hepatitis C virus (HCV). The HCV gene products can be viruses known to infect animals of any species, including, but not limited to, amphibians, reptiles, birds—such as stork, and heron, mice, hamsters, rats, rabbits, guinea pigs, woodchucks, pigs, micro-pigs, goats, dogs, cats, humans and non-human primates, e.g., baboons, monkeys, and chimpanzees. In certain embodiments, the injection device described herein can be used for the delivery of nucleic acids encoding proteins found in the hepatitis B virus (HBV). The HBV gene products can be viruses known to infect animals of any species, including, but not limited to, amphibians, reptiles, birds—such as stork, and heron, mice, hamsters, rats, rabbits, guinea pigs, woodchucks, pigs, micro-pigs, goats, dogs, cats, humans and non-human primates, e.g., baboons, monkeys, and chimpanzees.

In certain embodiments an adjuvant is used in addition to the active ingredient. For instance, a pharmacologic agent can be added to a drug being delivered by a device described herein as needed to increase or aid its effect. In another example, an immunological agent that increases the antigenic response can be utilized with a device described herein. For instance, U.S. Pat. No. 6,680,059, which is hereby incorporated in its entirety by reference, describes the use of vaccines containing ribavirin as an adjuvant to the vaccine. However, an adjuvant may refer to any material that has the ability to enhance or facilitate an immune response or to increase or aid the effect of a therapeutic agent.

In certain embodiments, any nucleic acid can be used with the device and methods presented, for example, plasmid DNA, linear DNA, antisense DNA and RNA. For instance, the nucleic acid can be a DNA expression vector of the type well known in the art. In some embodiments, the invention is used for the purpose of DNA or RNA vaccination. That is, the invention includes a method of enhancing the transmembrane flux rate of an injected DNA or RNA nucleic acid into the intracellular space.

In certain embodiments, the needles can be used for high pressure injection into various tissues of organisms, wherein it is desirable to deliver a therapeutic material. For instance, the tissue could be skeletal muscle, adipose tissue, an internal organ, bone, connective tissue, nervous tissue, dermal tissue, and others. For instance, DNA vaccines may delivered by intramuscular injection into skeletal muscle or by intradermal injection into the dermis of an animal. In other embodiments, a therapeutic material may be delivered via parenteral delivery into subcutaneous or intraperitoneal tissues. Depending on the target tissue and therapeutic agent or agents being delivered, parameters of the needles may be appropriately modified to accommodate the desired physical properties necessary to achieve generation of the pressure sufficient to enhance agent delivery.

In some embodiments, the injection device may be configured to deliver a therapeutic material at a predetermined delivery rate. For example, the syringe may controlled by a spring-actuated device that produces a desired stroke speed for pressing the syringe plunger to produce a desired delivery rate. U.S. Pat. No. 6,019,747 discloses one example of such a device and is hereby incorporated by reference in its entirety. Other configurations are known in the art and within the scope of the present application. The delivery rate may, for example, be at least 0.1 mL/s, 0.3 mL/s, 0.5 mL/s, 0.8 mL/s, 0.9 mL/s, 1.0 mL/s, 1.1 mL/s, 1.2 mL/s, 1.3 mL/s, 1.4 mL/s, 1.5 mL/s, 2.0 mL/s, or 3.0 mL/s The delivery rate may, for example, be no more than 20.0 mL/s, 10.0 mL/s, 7 mL/s, 6 mL/s, 5 mL/s, 4 mL/s, 3 mL/s, or 2 mL/s. As discussed further below, the present application includes methods of using the injection device. Accordingly, the method may include delivering a therapeutic material at a predetermined rate, such as any of the rates disclosed above.

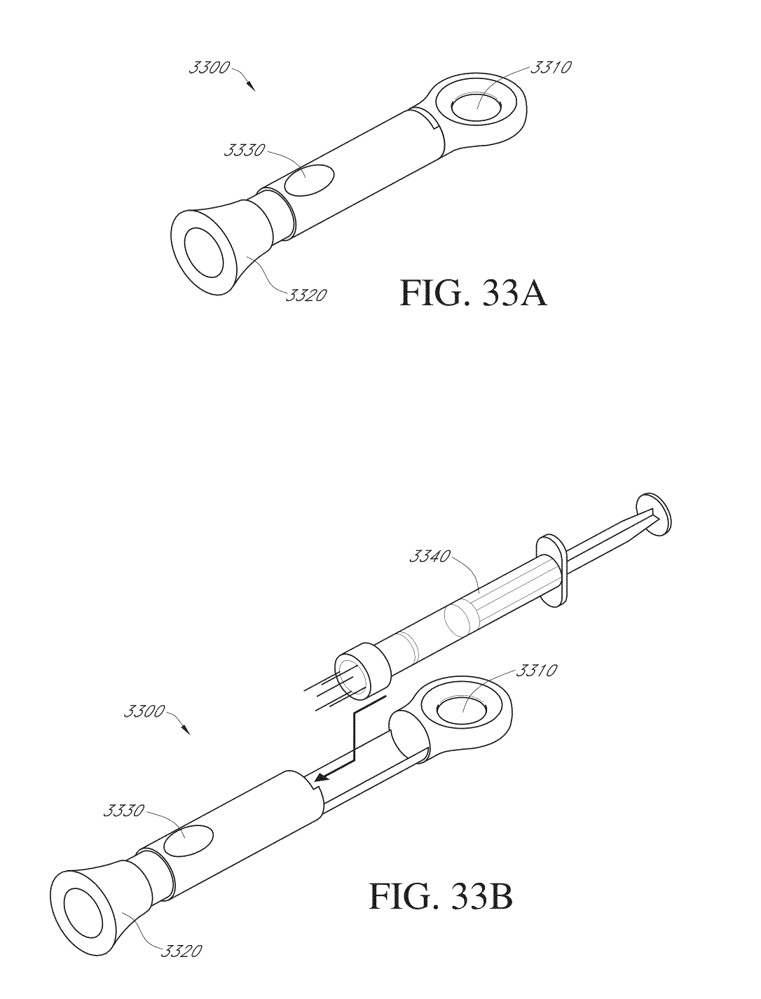

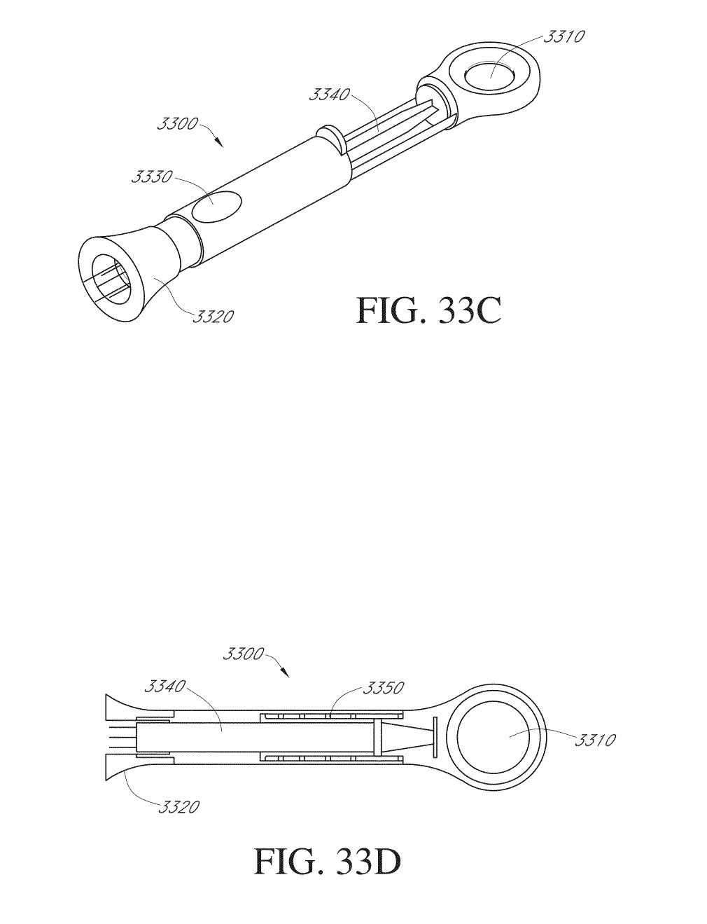

FIG. 33A is one example of spring-actuated device that can be used with the needles devices of the present applicant. Spring-actuated device 3300 includes loading ring grip 3310 on one side and depth adjusting member 3320 on an opposite side. Depth adjustment member 3320 may rotatably engaging spring-actuated device 3300 and be configured to adjust the depth that needles penetrate tissue when administering to a subject. Trigger button 3330 can be pressed to trigger the device to compress the needle plunger and inject therapeutic material. FIG. 33B shows needle device 3340 being inserted into spring actuated device 3300. Loading ring grip 3310 is withdrawn so that the needles can be inserted along the lumen of spring actuated device 3300. FIG. 33C shows needle device 3340 loaded within spring actuated device 3300. FIG. 33D shows a side view of spring-actuated device 3300 where springs 3350 are disposed along the lumen of spring-actuated device 3320 extending along a length of needle device 3340. Springs 3350 are configured to extend when loading ring grip 3310 is withdrawn and compress the plunger of syringe 3340 upon pressing trigger button 3330.

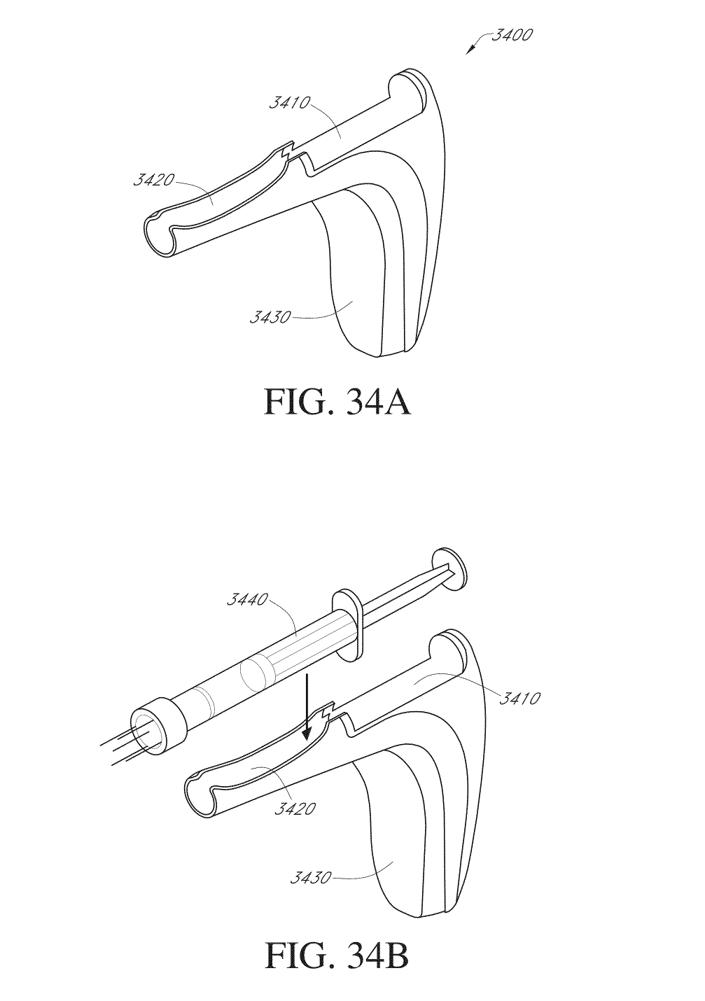

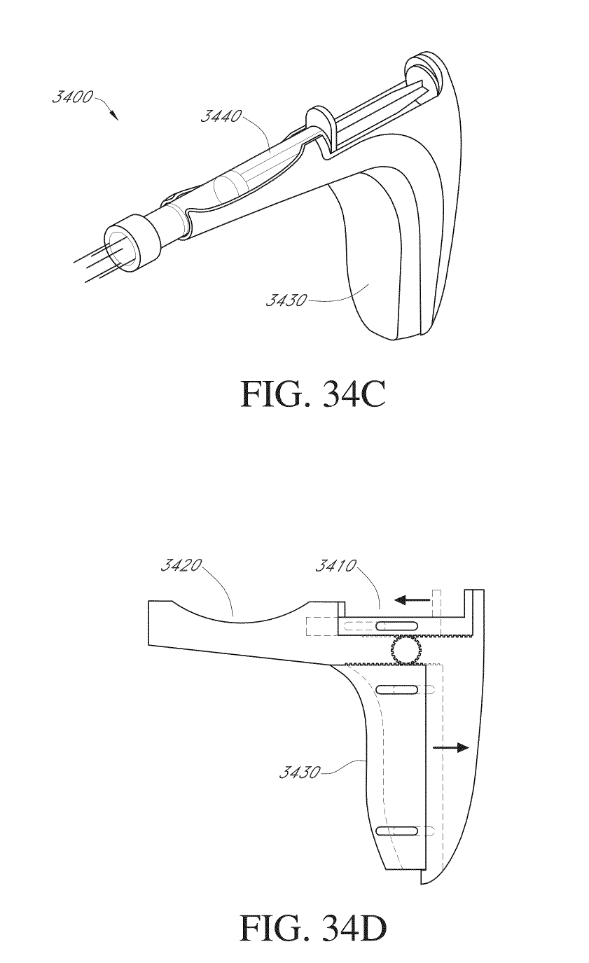

FIG. 34A is one example of a trigger device that can be used with the needle devices of the present application. Trigger device 3400 includes plunger aperture 3410 configured to receive the plunger portion of the syringe, and barrel aperture 3420 configured to receive the barrel portion of the syringe. Trigger 3430 is configured so that squeezing trigger 3430 depresses the plunger of a syringe. FIG. 34B shows needle device 3440 being inserted into trigger device 3400. FIG. 33C shows needle device 3440 loaded within trigger device 3400. FIG. 33D is a side view of trigger device 3400 where trigger 3430 is coupled to plunger aperture 3410 (e.g., coupled by a lever or gear) so that squeezing trigger 3430 compressed the plunger of the needle device and injects the therapeutic material.

Aspects of the invention also concern methods of making one or more of the aforementioned devices. By one approach, one or a plurality of the needles described herein are provided and said needle(s) are attached to a syringe that contains a therapeutic agent (e.g., a nucleic acid such as DNA, RNA, protein, or a compound). The attachment of the needle(s) and the syringe can be made such that the needle cannot be removed from the syringe (e.g., the needle and syringe are molded together) or the attachment can be made such that the needle and the syringe are detachable. Preferably, the attachment of the needle(s) and the syringe is done prior to loading the syringe with the therapeutic agent. The needle and syringe can be sterilized prior to or after adding the therapeutic agent. Preferably, the needle and syringe assembly is sterilized prior to addition of the therapeutic agent and shortly after sterilization, sterilized therapeutic agent is added in a sterile fashion. Desirable manufacturing processes are used to produce a single use device comprising one or more of the sterilized needles described herein, which are attached to one or more sterilized syringes that contain a single dose of one or more sterilized therapeutic agents. These single use devices can be separately sterile packaged such that a user merely needs to tear open a package and inject the therapeutic agent into a suitable tissue (e.g., single use DNA vaccination by injection into muscle).

Aspects of the invention also concern methods of using one or more of the aforementioned devices. By one approach, methods of intracellular delivery of a compound are provided, wherein a compound contained in a device described herein is administered to a subject. In some embodiments, a compound (e.g., a nucleic acid, such as DNA or protein) is provided in a device described herein (e.g., a syringe comprising one or more of the needles described herein). The compound is then delivered to the subject by inserting the needles into tissue of the subject, deploying the plunger to provide pressure on the solution in the syringe thereby pressing the compound out the apertures of the needles at a desired pressure. The increased pressure in the tissue promotes the uptake of the compound by the cells thereby allowing for the intracellular delivery of the compound. Indeed, any therapeutic material in which it is desirable for the material to be injected into under a high-injection pressure can be used in conjunction with the invention, including, but not limited to, polypeptides, carbohydrates, microparticles, steroids, or low-molecular weight molecules. For instance, nucleic acid and proteins can be simultaneously or serially introduced into an tissue undergoing high injection pressure.

Some embodiments concern methods of expressing a protein from DNA, wherein a device as described herein is provided (e.g., a syringe comprising one or more of the needles described herein and a DNA), the needles are inserted into a tissue of a subject (e.g., muscle), the DNA is introduced into the tissue by exiting the apertures under pressure (e.g., pressure exerted by deploying the plunger and pressing it toward the DNA solution in the syringe), and the DNA is taken up by the muscle cells. Optionally, the device containing the DNA is introduced or deployed in a manner that promotes an inflammatory response (e.g., mobilization of or activation of cells associated with an inflammatory response). Optionally, the needle design (e.g., plurality of apertures) or configuration of the device produces an inflammatory response (e.g., mobilization of or activation of cells associated with an inflammatory response). Optionally, the amount of protein expression and/or mobilization of cells associated with an inflammatory response is measured. Such measurements can be made using immunology and/or histochemistry.

Accordingly, some aspects of the invention concern methods of inducing an immune response to a desired antigen, whereby, a device as described herein is provided (e.g., a syringe comprising one or more of the needles described herein and a DNA), the needles are inserted into a tissue of a subject (e.g., muscle), the DNA is introduced into the tissue by exiting the apertures under pressure (e.g., pressure exerted by deploying the plunger and pressing it toward the DNA solution in the syringe), and the DNA is taken up by the muscle cells. Subsequently, protein encoded by the DNA is made in the cells, and the immune system responds to the protein. Optionally, an immune response to the antigen produced from the introduced DNA is measured (e.g., presence of antibody, specific T cells, or reduction or clearance of infection).

Using certain embodiments of the invention, gene constructs may be administered directly into a skeletal muscle tissue for the uptake of the gene by a cell for the subsequent synthesis of the encoded product. In some methods of the invention, a high-pressure injection needle may be used to propel a liquid that contains DNA or RNA molecules into a subject's tissue. The liquid is propelled at a sufficient velocity such that upon impact with the tissue the liquid exerts a high pressure onto the tissue, increasing cell permeability, and causing the DNA or RNA molecule to permeate the cells in the area. In some embodiments, a high-pressure injection needle may be used to deliver genetic material to tissue of other organs in order to introduce a nucleic acid molecule to cells of that organ. Indeed, it will be readily recognized that other gene delivery mechanisms well known in the art can be adapted to be used with embodiments of the present invention, including liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736, all of which are hereby included in their entirety by reference). Additionally, one may use a variety of adjuvants (e.g., ribavirin), to either enhance immunogenicity and/or cell permeability.

For instance, by way of example only and not by way of any limitation, certain embodiments of the invention can be used in conjunction with the constructs described in U.S. Publication Number 2005-0277192 and U.S. Publication Number 2005-0124573, the entireties of which are hereby expressly incorporated by reference. These references describe the use of a nucleic acid encoding hepatitis C virus (HCV) nonstructural protein 3/4A (NS3/4A) to promote an immune response in humans. For example, it was observed that when HCV NS3/4A gene was transfected into mammalian cells, vis a vis a eukaryotic expression vector, appreciable levels of expression of NS3 were observed. Further, mice immunized with the NS3/4A gene were found to have primed high levels of NS3-specific antibodies and antigen specific T cells. Recently, similar constructs have been found to produce a potent immune response in clinical trials with patients that are infected with HCV.

Accordingly, some embodiments concern methods of treating and preventing HCV infection, wherein one or more of the devices described herein, which contain one or more of the HCV DNA constructs that have been shown to produce a potent immune response in humans, is provided to a patient that is infected with or who is at risk of infection by HCV. Optionally, an individual in need of a medicament that prevents and/or treats HCV infection is identified and said individual is then provided a medicament comprising one or more of the HCV constructs that have been found to produce a potent immune response in humans (e.g., an expression construct encoding NS3/4A) using a high-pressure injection needle device, as described herein. Optionally, an immune response to NS3/4A, a reduction in viral titer, or production anti-HCV antibodies is measured in the inoculated individual after treatment or during the course of treatment.

However, the current invention is not limited to antigens of HCV for DNA immunization. Indeed, the invention can be used any time in which expression of any antigenic peptide within cell is desirable. For instance, some non-limiting examples of known antigenic peptides in relation to specific disease states include the following:

HBV: PreS1, PreS2 and Surface env proteins, core and pol

HIV: gp120, gp40, gp160, p24, gag, pol, env, vif, vpr, vpu, tat, rev, nef

Papilloma: E1, E2, E3, E4, E5, E6, E7, E8, L1, L2

HSV: gL, gH, gM, gB, gC, gK, gE, gD, ICP47, ICP36, ICP4

as taught in U.S. Pat. No. 7,074,770 to Charo, et al., entitled “Method of DNA vaccination,” and which is hereby incorporated by reference in its entirety. Some of the embodiments described herein also include and/or administer one or more of the nucleic acids selected from the group consisting of: mRNA, tRNA, rRNA, cDNA, miRNA (microRNA), siRNA, (small interfering RNA), piRNA (Piwi-interacting RNA), aRNA (Antinsense RNA), snRNA (Small nuclear RNA), snoRNA (Small nucleolar RNA), gRNA (Guide RNA), shRNA (Small hairpin RNA), stRNA (Small Temporal RNA), ta-siRNA (Trans-acting small interfeing RNA), cpDNA, (Chloroplast DNA), gDNA (Genomic DNA), msDNA (Multicopy single-stranded DNA), mtDNA (Mitochondrial DNA), GNA (Glycol nucleic acid), LNA (Locked nucleic acid), PNA (Peptide nucleic acid), TNA (Threose nucleic acid), Morpholino contaiing nucleic acids, sulfur-containing nucleic acids, 2-O-methyl nucleic acids, and nucleic acids containing one or more modified bases or spacers.

By one approach, for example, in a first study, HCV infected individuals are injected with a solution containing approximately 6.0 ml 0.9% NaCl containing approximately 0.25 mg/kg bodyweight of ChronVac-C (coNS3/4A DNA), an expression plasmid encoding codon-optimized HCV NS3/4A, in the thigh muscle using a large high injection pressure (HIP) injector. In a second study, HBV infected individuals are injected with a solution containing approximately 6.0 ml 0.9% NaCl containing approximately 0.25 mg/kg bodyweight of coHBcAg (an expression plasmid encoding codon-optimized HBV core antigen) in the thigh muscle using a large HIP injector. The large HIP injector has 4 needles oriented in a triangular formation, equally spaced with 6 mm between each needle. The center needle is placed in the middle of the equilateral triangle formed by the three outer needles. Each needle of the large HIP injector has 10 apertures. The outer needles all have apertures opening to the center and the center needle has apertures opening at four directions at 90 degree angles.

At day 5 and 10 blood is drawn from the inoculated individuals, peripheral blood mononuclear cells (PBMCs) are isolated, and the PBMCs are analyzed for T cell proliferation. The PBMCs can be assayed for in-vitro proliferative recall responses using a standard 96 h proliferation assay. (See Lazinda et al., J. Gen. Virol. 82:1299-1308 (2001), herein expressly incorporated by reference in its entirety.) In brief, microtiter plates are seeded with approximately 200,000 cells/well and the cells are incubated with media alone or recombinant NS3 or HBcAg. PBMCs are also incubated with Concanavalin A (ConA) as a positive control. After 72 hours, radioactive thymidine is added and 16-24 hours later the cells are harvested. The radioactivity of the cells as counts per minute are measured. Additionally, the presence of antibodies specific for NS3/4A and or HBcAg can be determined using standard assays (e.g., ELISA). Optionally, a boost injection is provided at two or three week intervals. The results will show that humans immunized with the large HIP injector show appreciable immune response to NS3/4A and/or HBcAg.

The following examples are given to illustrate various embodiments of the present invention in the field of DNA immunization, which can be delivered to a subject in need of an immune response to the antigen contained therein. It is to be understood that the following examples are not comprehensive or exhaustive of the many types of embodiments which can be prepared in accordance with the present invention.

### Example 1

New Zealand white rabbits weighing 3.5 Kg were injected with a solution containing 0.3 ml 0.9% NaCl containing 0.9 mg of either ChronVac-C (coNS3/4A DNA) or coHBcAg in the tibialis anterior using either a large high injection pressure (HIP) injector, a small HIP injector, or a regular 27 gauge needle. Rabbits were injected either in the right tibialis anterior, left tibialis anterior, or both.

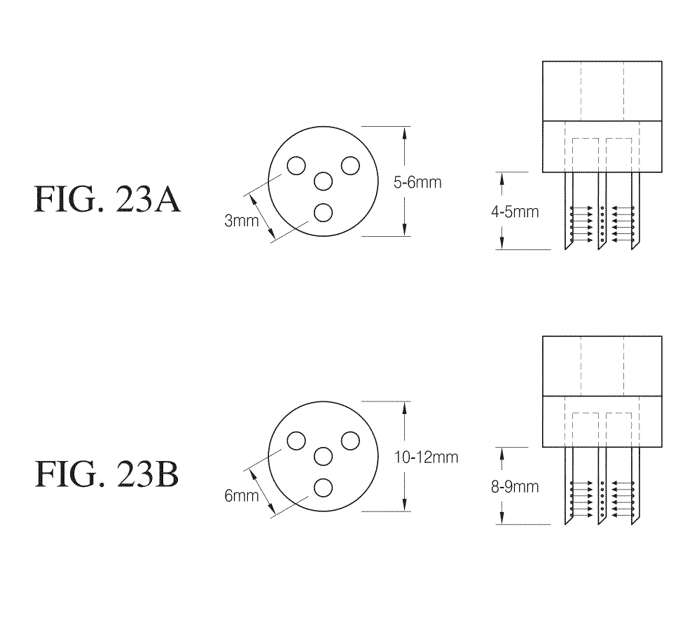

As described in FIG. 23A, the small HIP injector has needles 4-5 mm in length. The small HIP injector has 4 needles. As depicted in the figure, the three outer needles are oriented in a triangular formation, equally spaced with approximately 3 mm between each needle to form an equilateral triangle. The center needle is placed in the middle of the triangle formed by the three outer needles. Each needle has 6 apertures. The outer needles all have apertures opening to the center and the center needle has apertures opening at four directions at 90 degree angles. The large HIP injector (FIG. 23B) has needles 8-9 mm in length. The large HIP injector has 4 needles oriented in a triangular formation, equally spaced with 6 mm between each needle. The center needle is placed in the middle of the equilateral triangle formed by the three outer needles. Each needle of the large HIP injector has 10 apertures. The outer needles all have apertures opening to the center and the center needle has apertures opening at four directions at 90 degree angles. The injection scheme is shown in table 1 below:

TABLE 1

| Rabbit | Needle | Injection | | | |
|---|---|---|---|---|---|
| # | Type | Site | Plasmid | Dose | Sacrificed |
| 115 | HIP-large | Right TA | coNS3/4A | 0.9 mg/0.3 ml | Day 5 |
| | Regular | Left TA | coNS3/4A | 0.9 mg/0.3 ml | |
| | Needle | | | | |
| 116 | HIP-large | Right TA | coNS3/4A | 0.9 mg/0.3 ml | Day 5 |
| | Regular | Left TA | coNS3/4A | 0.9 mg/0.3 ml | |
| | Needle | | | | |
| 117 | HIP-small | Right TA | coNS3/4A | 0.9 mg/0.3 ml | Day 5 |
| | None | — | — | — | |
| 118 | HIP-small | Right TA | coNS3/4A | 0.9 mg/0.3 ml | Day 5 |
| | None | — | — | — | |
| 119 | HIP-large | Right TA | coNS3/4A | 0.9 mg/0.3 ml | Day 10 |
| | HIP-large | Left TA | coHBcAg | 0.9 mg/0.3 ml | |
| 120 | HIP-large | Right TA | coNS3/4A | 0.9 mg/0.3 ml | Day 10 |
| | HIP-large | Left TA | coHBcAg | 0.9 mg/0.3 ml | |
| 121 | Regular | Right TA | coNS3/4A | 0.9 mg/0.3 ml | Day 10 |
| | Regular | Left TA | coHBcAg | 0.9 mg/0.3 ml | |
| 122 | None | — | — | — | Day 10 |
| | none | — | — | — | |

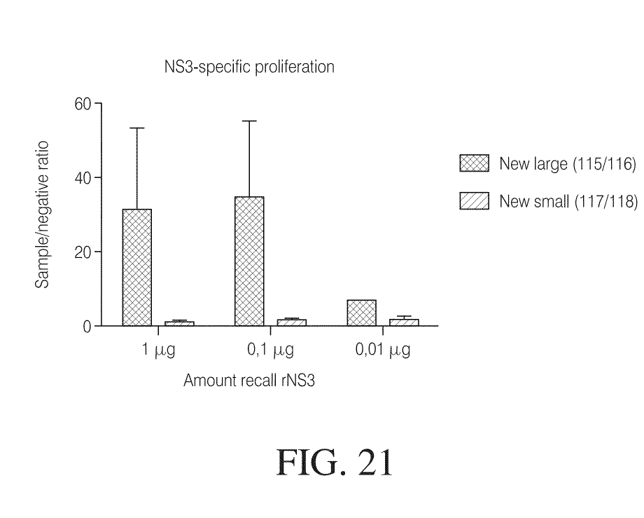

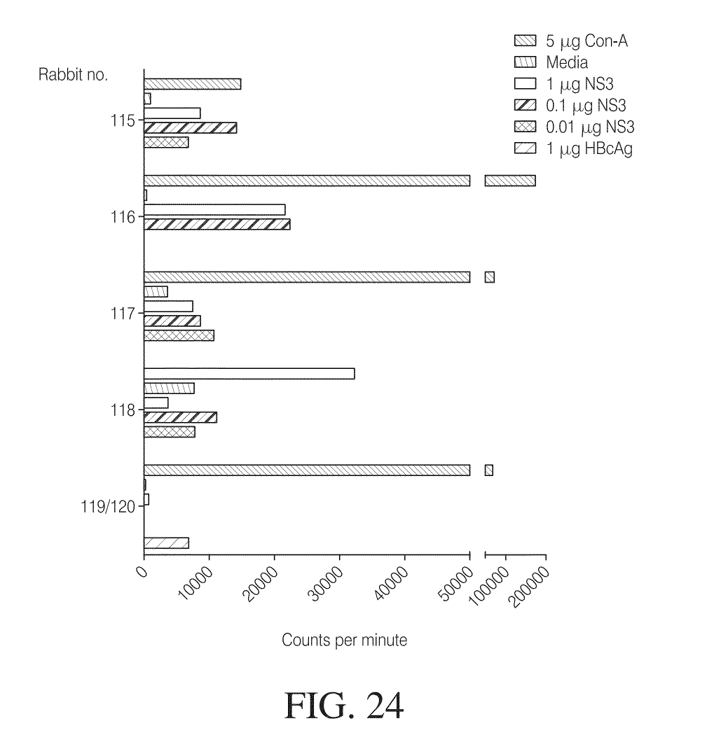

At day 5, rabbits 115-118 were sacrificed and peripheral blood mononuclear cells (PBMCs) were analyzed for T cell proliferation. The PBMCs were assayed for in-vitro proliferative recall responses using a standard 96 h proliferation assay. (See Lazinda et al., J. Gen. Virol. 82:1299-1308 (2001), herein expressly incorporated by reference in its entirety.) In brief, microtiter plates were seeded with approximately 200,000 cells/well and the cells were incubated with media alone, recombinant NS3 or HBcAg. PBMCs were also incubated with Concanavalin A (ConA) as a positive control. After 72 hours, radioactive thymidine was added and 16-24 hours later the cells were harvested. The radioactivity of the cells as counts per minute are depicted in FIG. 24 and listed in TABLE 2. The proliferation was determined as radioactivity of the cells as the counts per minute (cpm) of cells incubated with the antigen divided by the CPM of the cells incubated with the media alone (sample to negative ration; S/N). The results are shown in FIG. 21.

TABLE 2

| | 5 μg | | 1 μg | 0.1 μg | 0.01 μg | 1 μg |
|---|---|---|---|---|---|---|
| Rabbit | Con-A | media | NS3 | NS3 | NS3 | HBcAg |
| 115 | 14792 | 958 | 8570 | 14141 | 6816 | Not tested |
| 116 | 172935 | 406 | 21595 | 22360 | Not tested | Not tested |
| 117 | 71133 | 3632 | 7465 | 8625 | 10658 | Not tested |
| 118 | 32152 | 7632 | 3705 | 11152 | 7724 | Not tested |
| 119/120 | 67470 | 191 | 717 | Not tested | Not tested | 6838 |

The results show that rabbits immunized with the large HIP injector show a more robust immune response displayed through greater T cell proliferation than rabbits immunized with the small HIP injector. The data also provide strong evidence that the DNA that was introduced into the muscle tissue by the HIP injectors was effectively transferred into the cell, wherein it was transcribed, translated, and was used by the immune system of the animal to generate a potent immune response. Both the DNA encoding the HCV antigen NS3/4A and the DNA encoding the HBV antigen HBcAg effectively generated a potent immune response in mammals demonstrating that a variety of DNAs that encode immunogens can be effectively introduced into mammals using a delivery device described herein to induce an immune response in the inoculated animal.

The injection site for each rabbit was also collected for histological evaluation (as described in Ahlen et al., In Vivo Electroporation Enhances the Immunogenicity of Hepatitis C Virus Nonstructural 3/4A DNA by Increased Local DNA Uptake, Protein Expression, Inflammation and Infiltration of CD3+ T Cells. J. Immunol. 2007 179(7):4741-53, herein incorporated by reference in its entirety). Briefly, the tissue was fixed in a buffered 4% formaldehyde solution, dehydrated, and embedded in paraffin. The embedded tissues were sectioned in 4-6 μm sections. The sections were mounted onto glass slides and stained with hematoxylin and eosin stain (H&E), or polyclonal mouse sera from a coNS3/4A DNA-immunized mouse, which was detected by a biotinylated goat anti-mouse secondary antibody and peroxidase labeled streptavidin using an insoluble peroxidase substrate.

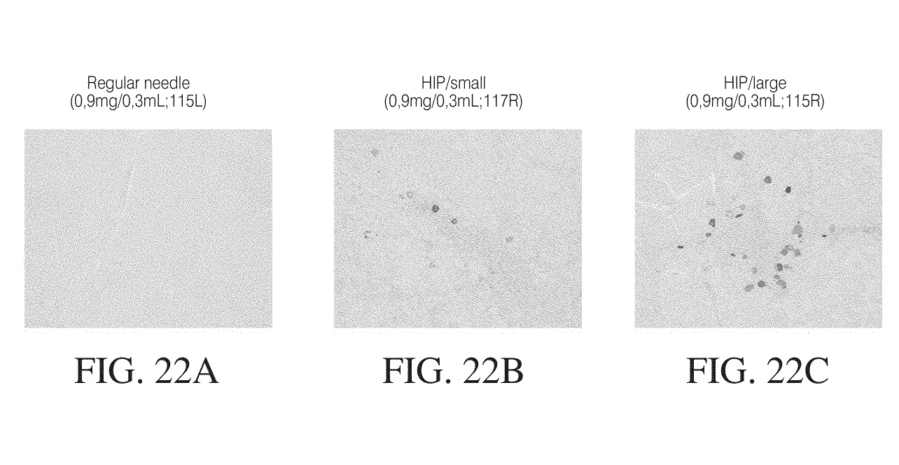

The results are shown in FIG. 22A-C. The injection of 0.9 mg of coNS3/4A with both HIP injectors produced significant amounts of local inflammation, regeneration, and fibrosis, as indicated by the high concentration of stained immune cells that localized to the injection site, in particular, between the needles. The data show that the large injector produced a better inflammatory response than the small injector in the rabbits. The injection of 0.9 mg of coNS3/4A with the conventional 27 gauge needle caused very little local inflammation, regeneration, and fibrosis, as indicated by the almost absent stained immune cells localized to the injection site. Additionally, both the HIP injectors induced the cells surrounding the injection site to produce significant amounts of NS3 protein, as indicated by the antibody labeling; whereas, the conventional injection with the 27 gauge needle under these conditions produced no detectable NS3 protein. Accordingly, the data show that the HIP injectors effectively delivered DNA into the cells, wherein it was transcribed and translated in significant amounts, which could be detected by an antibody specific for NS3 but the conventional injection with the 27 gauge needle did not.

The results provided in this example demonstrate that the HIP injectors described herein effectively deliver an expression plasmid that encodes an antigen into a cell of a subject in quantities sufficient to allow for a level of protein expression that is detectable by an antibody directed to the antigen and in an amount that is sufficient to generate appreciable amounts of antigen-specific T cells. That is, the data show that the HIP injectors described herein effectively deliver nucleic acids to cells of the body in an amount sufficient to produce a potent immune response in the subject. Thus, injecting a DNA vaccine using the HIP injector improves the immune response relative to standard methods of delivering vaccines.

### Example 2

The mechanisms by which a high injection pressure (HIP) needle improves the potency of intramuscular DNA vaccination are characterized by using the hepatitis C virus nonstructural (NS) 3/4A gene. Sustained control and clearance of HCV infection is related to an effective immune response, in particular a T cell response targeted to the nonstructural NS3 protein. By activating T cells outside the liver via vaccination, one may allow for the complementing or reshaping of the existing T cell repertoire. The present NS3/4A plasmid-based vaccine example is tested in mice. In vivo HIP needle administered vaccine is contemplated to increase the permeability of myocyte cell members, wherein the plasmid is effectively taken up in the nucleus and expressed, thereby inducing a functional in vivo immune response. The use of an in vivo HIP needle enhances the immunogenicity of coNS3/4A by both increasing protein expression levels and the duration of expression and by enhancing the infiltration of CD3+ T cells and a local inflammatory response at the site of injection.

Male and female C57BL/6 mice are bred and caged at five mice per cage. The mice are fed a commercial diet (RM3 (p) PL IRR diet; Special Diet Service) with free access to food and water. All animals are at least 6 weeks of age before start of the experiment. The SV40-luciferase plasmid (pGL4.13-[Luc2-5V40]; Promega) is produced in-house by standard technologies. The coNS3/4A plasmid is produced under Good Manufacturing Practice regulations.

The coNS3/4A DNA vaccine is administered by a single intramuscular injection (0.05 ml in mice) with a two-barrel 27-gauge HIP needle into the right tibialis anterior (TA) muscle. Doses range from 0.5 to 50 μg of DNA in mice. One two-barrel needle is used per injection and per animal. The procedure is repeated up to three times in mice at monthly intervals.

Detection of mouse antibodies to NS3 by enzyme immunoassay is performed using standard immunoassay techniques. Antibodies titers are determined as the last serum dilution giving an OD at 405 nm of three times the OD at the same dilution of a non-immunized animal serum. With respect to NS3 antibody levels, a dose-response relationship is seen after vaccination with different doses of coNS3/4A-DNA administered with or without using the HIP needle. The boost effect is seen after immunization. The smaller dose given with the HIP needle induces the same mean NW-specific antibody levels as a greater dose delivered without the HIP needle. In conclusion, the HIP needle makes the coNS3/4A DNA-based immunization more effective with respect to antibody responses, supporting the benefits of the adjuvant effects mediated by using a HIP needle.

### Example 3

New Zealand White rabbits weighing 2.5-3.5 kg, are purchased from commercial vendors. The coNS3/4A DNA vaccine is administered by a single intramuscular injection with a four-barrel 27-gauge HIP needle into the right tibialis anterior (TA) muscle. Doses range from 70 to 700 μg of DNA. One four-barrel needle is used per injection and per animal. The procedure is repeated up to five times in rabbits at monthly intervals.

Detection of rabbit antibodies to NS3 by enzyme immunoassay is performed using standard immunoassay techniques. Antibodies titers are determined as the last serum dilution giving an OD at 405 nm of three times the OD at the same dilution of a non-immunized animal serum.

Proliferative responses to NS3 are determined in rabbit whole blood. A total of 4 ml of whole blood is obtained from the ear artery of each rabbit immediately before the first vaccination and 2 weeks after each vaccination and collected in heparin tubes. Plasma and peripheral mononuclear cells (PMBC) are isolated by gradient centrifugation. Plasma is stored at −80° C. until the analysis of NS3-specific antibody by enzyme immunoassay. PBMCs are immediately assayed for in vitro proliferative recall responses using a standard 96 hour proliferation assay. In brief, microplates are seeded with 200,000 cells per well and the cells are incubated with medium alone, ConA, PHA, or rNS3. After 72 hours, radioactive thymidine is added and 16-24 hours later, the cells are harvested. Proliferation is determined from the radioactivity of the cells as the counts per minute (cpm) of cells incubated with an antigen divided by the cpm of the cells incubated with medium alone, sample to negative (S/N) ratio. Groups are compared by the mean S/N ratios at each time point.

Rabbits are injected in the right TA with 300 μl of saline containing the indicated amount of coNS3/4A DNA. Antibody levels are recorded as the mean end point titers. Peak antibody end point titers are reached after several injections.

Data is recorded showing the dose-response relation with respect to induction of NS3-specific proliferative responses in PBMC in rabbits immunized using a HIP needle. Data is collected to indicate a proliferative result as the mean S/N of duplicate or triplicate determinations in the presence of rNS3 in vitro.

NS3-specific proliferation will be detectable. The mean NS3-recalled proliferation is consistently higher in the groups receiving higher doses of coNS3/4A DNA as compared with the control group. Thus, the vaccination primes in vitro detectable T cell responses in rabbits.

### Example 4

In a next series of experiments, the injection needle(s) described herein are modified for use with existing gene transfer technologies, including gene gun delivery systems (see e.g., U.S. Pat. Nos. 5,036,006; 5,240,855; and 5,702,384, the disclosures of which are hereby expressly incorporated by reference in their entireties), delivery systems using electroporation (see e.g., U.S. Pat. Nos. 6,610,044 and 5,273,525, the disclosures of which are hereby expressly incorporated by reference in their entireties) and microneedle delivery systems (see e.g., U.S. Pat. Nos. 6,960,193; 6,623,457; 6,334,856; 5,457,041; 5,527,288; 5,697,901; 6,440,096; 6,743,211; and 7,226,439, the disclosures of which are hereby expressly incorporated by reference in their entireties). In these experiments, the NS3/4A-pVAX1 vector is administered to mice or rabbits via the modified gene gun delivery system, the modified electroporation device, or the modified microneedle delivery system. Purified NS3/4A-pVAX1 vector is used to immunize groups of mice or rabbits. The plasmid is injected directly into regenerating tibialis anterior (TA) muscle via either the modified gene gun delivery system, the modified electroporation device, or the modified microneedle delivery system. Immunization of is performed with approximately 0.25 mg/kg of DNA of plasmid DNA. Immunizations are performed on weeks 0, 4, and 8.

Enzyme immunosorbent assays (EIAs) are used to detect the presence of murine N53-specific antibodies. These assays are performed essentially as described (Chen et al., Hepatology 28(1): 219 (1998)). Briefly, rNS3 is passively adsorbed overnight at 4° C. to 96-well microtiter plates (Nunc, Copenhagen, Denmark) at 1 μg/ml in 50 mM sodium carbonate buffer (pH 9.6). The plates are then blocked by incubation with dilution buffer containing PBS, 2% goat serum, and 1% bovine serum albumin for one hour at 37° C. Serial dilutions of mouse sera starting at 1:60 are then incubated on the plates for one hour. Bound murine and rabbit serum antibodies are detected by an alkaline phosphatase conjugated goat anti-mouse or goat anti-rabbit IgG (Sigma Cell Products, Saint Louis, Mo.) followed by addition of the substrate pNPP (1 tablet/5 ml of 1M Diethanol amine buffer with 0.5 mM MgCl₂). The reaction is stopped by addition of 1M NaOH and absorbency is read at 405 nm.

After four and six weeks, all mice and rabbits immunized with NS3/4A-pVAX1 will develop NS3 antibodies. Similarly, all mice and rabbits immunized with NS3/4A-pVAX1 will develop potent T cell responses. All mice and rabbits immunized with NS3/4A-pVAX1 via either the modified gene gun delivery system, the modified electroporation device, or the modified microneedle delivery system will develop a potent immune response to the desired antigen.

### Example 5

A major obstacle that limits the efficacy of gene transfer and genetic vaccination in large animals including humans is the poor uptake of naked nucleic acid. Devices such using particle bombardment and in vivo electroporation has been developed and can improve on the poor uptake of nucleic acid in humans. However, these require either moving parts of electricity that limits the ease by which they can be used. We have therefore developed a simple injections needle that takes advantage of the fact that pores opens in cellular membranes when the hydrostatic pressure in the tissue increases. The basic design uses 3 to 10 circularly oriented needles where the ends of the needles have been sealed by laser welding. New openings of various sizes have been made on the needle shaft that direct the injected liquid centrally in the circle of needles. Finally one or more needles have been positioned centrally with openings in all directions. We can show that injection of a naked DNA plasmid in rabbit tibialis anterior muscle leads to an improved in vivo transfection of muscle fibres that express the transferred gene. In addition, T cell responses to the expressed transgene can be detected already after five days Importantly, this new needle can be used with any commercially available syringe and does not require and advanced skills in injection technologies. Thus, these new needles, termed In vivo Intracellular Injections Needle (IvIn) technology, offers a simple solution to gene transfer in vivo in large animals, hopefully also including humans.

### Example 6

It is well known that the exogenous capsid protein (HBcAg) of the hepatitis B virus (HBV) is highly immunogenic on a CD4+ T cell level in all species tested. However, HBcAg has not been explored as an adjuvant for genetic vaccines, and in particular the non-human forms of HBcAg. A key feature of using non-human HBcAg is that HBV is a very common infection that affects almost a third of the worlds population. Thus, HBcAg sequences from highly distant species should be used in order to be able to use these vaccines also in areas highly endemic for HBV. We here explored the use of HBcAg as a DNA vaccine adjuvant. We found that HBcAg-sequences effectively improved the immunogenicity of hepatitis C virus derived genes supporting that HBcAg can act as a intracellular adjuvant (iac). Importantly, the major role of the addition of HBcAg-sequences were seen in models mimicking the human HCV infection. HBcAg-based vaccines could overcome the profound T cell tolerance in transgenic mice co-expressing the human leucocyte antigen (HLA)-A2 and the HCV non-structural (NS) 3/4A complex. Here the presence of “healthy” non-tolerized heterologous T cells aided in the activation of the dysfunctional HCV NS3/4A-specific T cells. Thus, HBcAg effectively acts as an intracellular adjuvant that can help restoring a dysfunctional T cell response in a host with persistent presence of a viral antigen, as generally seen in chronic viral infections.

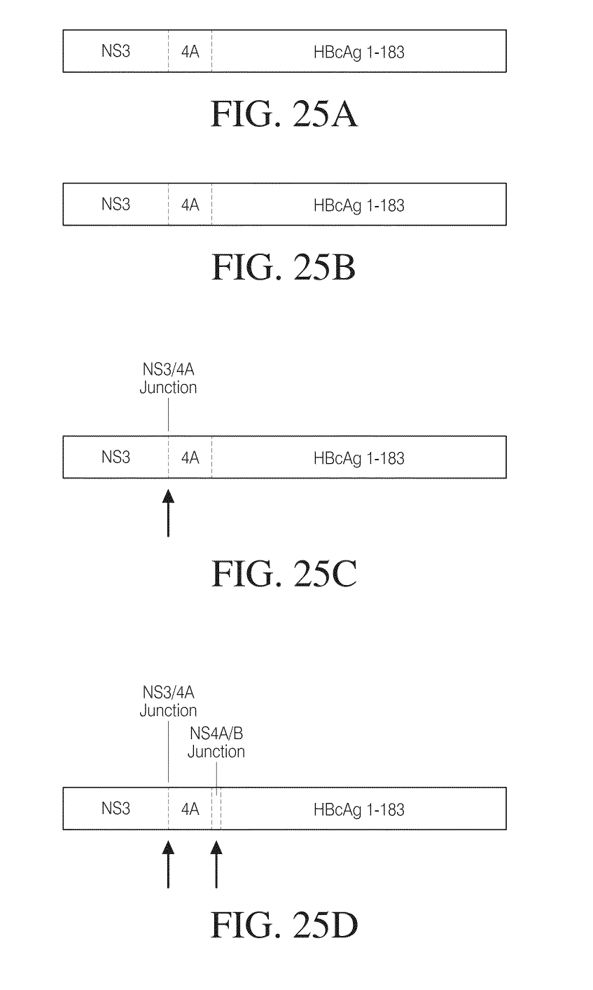

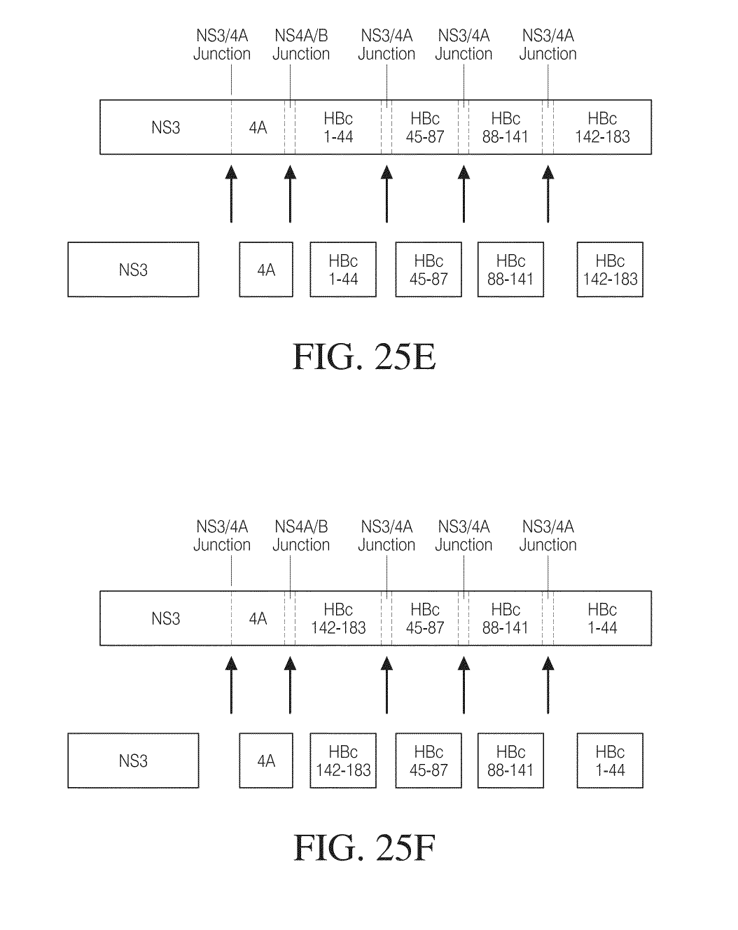

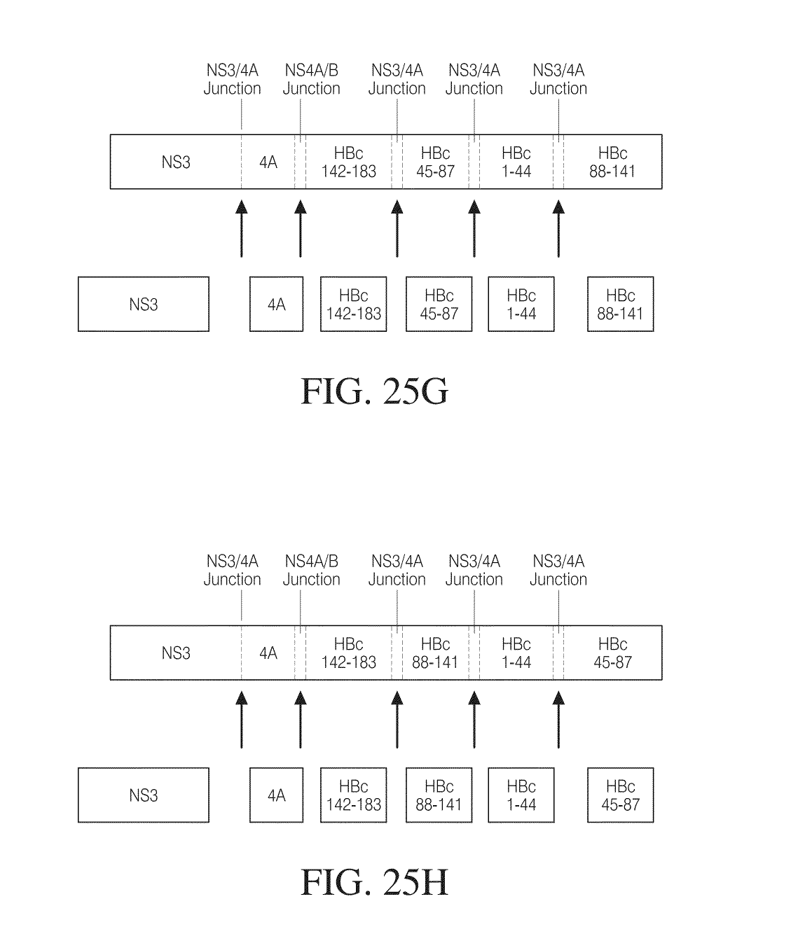

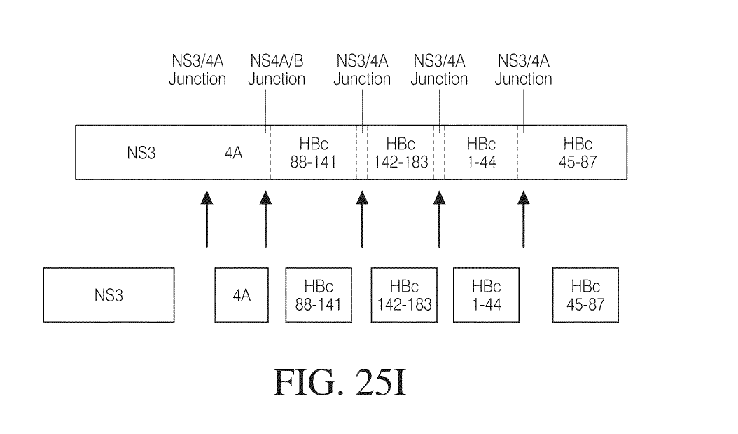

Some embodiments include, for example, one or more of the HBcAg nucleic acid or protein sequences disclosed in International Patent Application Publication Number WO 2009/130588, which designated the United States and was published in English, the disclosure of which is hereby expressly incorporated by reference in its entirety. Some embodiments include the NS3/4A/HBcAg fusions or a nucleic acid encoding said fusion identified in FIGS. 25 A-I, or a nucleic acid or a nucleic acid or a nucleic acid encoding a protein described in SEQ. ID NOS 1-32. Additional nucleic acid sequences encoding antigenic peptides, such as those described in WO 2009/130588 (e.g., birch antigen) and WO 2010/086743, both of which designated the United States and published in English, the disclosure of which is hereby expressly incorporated by reference in their entirety can also be joined to an HBcAg encoding nucleic acid sequence and said fusions can be administered to a subject in need thereof using one or more of the injection devices described herein. Some embodiments also include additional adjuvants, including but not limited to ribavirin or a CPG nucleotide e.g., SEQ. ID NO. 33. Any of the aforementioned embodiments can be incorporated into one or more of the injection devices described herein and can be administered to a subject in need thereof.

### Example 7

The force requirements for injecting material using an injection needle described herein were studied. Placebo liquid was injected into open space or chicken breast and the applied forces were measured using a Lloyd force tensometer.

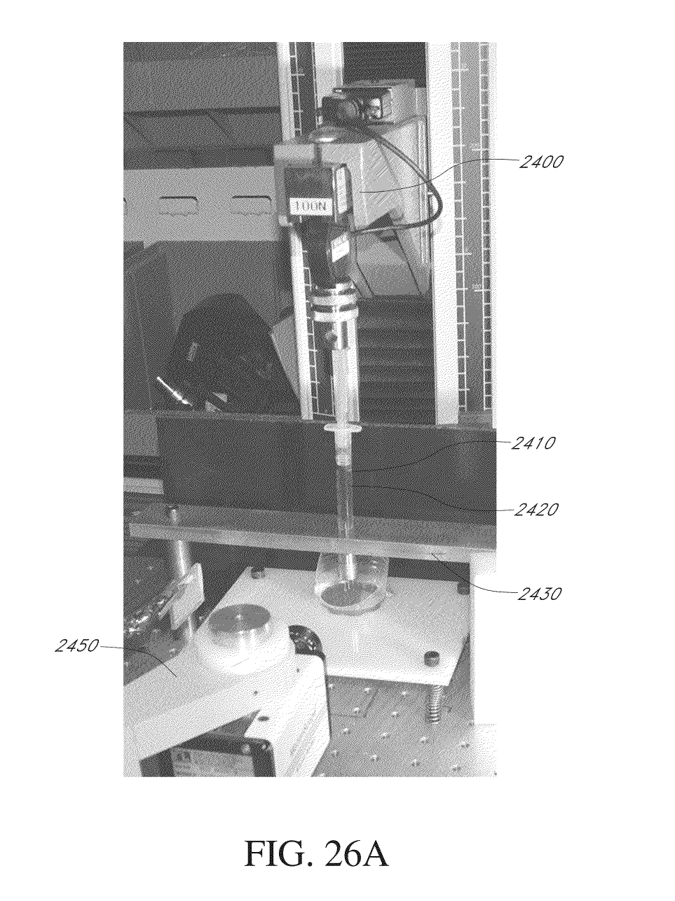

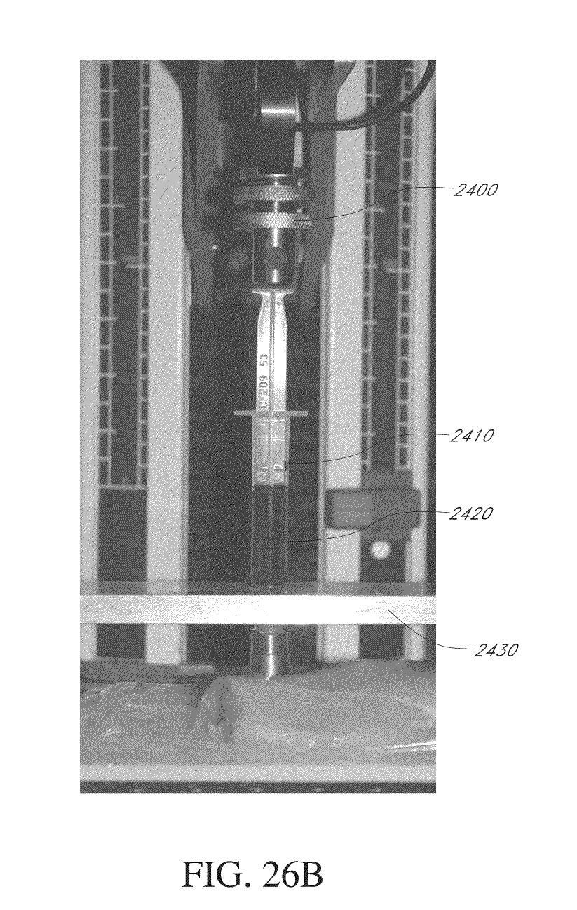

FIG. 26A is an example of the setup for measuring the force requirements when injecting material using one of the injection needle devices disclosed herein. Lloyd Force Tester 2400 was used to compress syringe 2410 containing fluid 2420 at a predetermined velocity to measure the applied force while injecting 0.3 mL of fluid (e.g., air or water). Support jig 2430 secured syringe 2410 during compression and high-speed camera 2440 recorded the spray pattern from the needles barrels 2450. Two different syringes were tested: (i) a 3 mL syringe requiring a plunger depth of 5.09 mm to inject 0.3 mL, and (ii) a 5 mL syringe requiring a plunger depth of 2.63 mm to inject 0.3 mL. An initial test studied the force required for injecting air into an open area (i.e., not positioned within muscle tissue). Tests were also completed for injecting died water into an open area or into chicken breast (e.g., as depicted in FIG. 26B).

The tested injection device include four needles configured with generally the same structure depicted in FIG. 8B. The length L₆ was 6 mm Needle 820b includes three zones, each having 15 apertures that all face one of the adjacent needles 820a, 820c, and 820d. That is, needle 820b include a first zone having 15 apertures that all face needle 820a, a second zone having 15 apertures that all face needle 820b, and a third zone having 15 apertures that all face needle 820c. Meanwhile, needles 820a, 820c, and 820d each include one zone of 15 apertures that all face needle 820b. All of the apertures in a given zone were spaced vertically apart along the axis of the needle barrel. Each aperture was separated by distance of about 0.2 mm between the centers of each apertures. Needles with 0.05 mm circular apertures or 0.1 mm circular apertures were tested.

The results are shown Table 3.

TABLE 3

| | Aper- | | Compres- | | | | Maxi- |
|---|---|---|---|---|---|---|---|
| | ture | Syringe | sion | Flow | | | mum |
| | Size | Volume | Speed | Rate | | Target | Force |
| Test | (mm) | (mL) | (mm/s) | (mL/s) | Fluid | Material | (N) |
| 1 | 0.1 | 3 mL | 17 | 1.0 | Air | None | 2.9 |
| 2 | 0.1 | 3 mL | 10.2 | 0.6 | Air | None | 2.6 |
| 3 | 0.1 | 3 mL | 5.1 | 0.3 | Air | None | 2.1 |
| 4 | 0.1 | 3 mL | 17 | 1.0 | H₂O | None | 16.0 |
| 5 | 0.1 | 3 mL | 10.2 | 0.6 | H₂O | None | 8.5 |
| 6 | 0.1 | 3 mL | 5.1 | 0.3 | H₂O | None | 4.0 |
| 7 | 0.1 | 3 mL | 17 | 1.0 | Died | Chicken | 18.0 |
| | | | | | H₂O | | |
| 8 | 0.1 | 3 mL | 10.2 | 0.6 | Died | Chicken | 9.8 |
| | | | | | H₂O | | |
| 9 | 0.1 | 3 mL | 5.1 | 0.3 | Died | Chicken | 5.25 |
| | | | | | H₂O | | |
| 10 | 0.1 | 5 mL | 17 | 1.9 | Air | None | 1.9 |
| 11 | 0.1 | 5 mL | 10.2 | 1.2 | Air | None | 1.2 |
| 12 | 0.1 | 5 mL | 5.1 | 0.6 | Air | None | 0.6 |
| 13 | 0.1 | 5 mL | 17 | 1.9 | H₂O | None | 36.0 |
| 14 | 0.1 | 5 mL | 10.2 | 1.2 | H₂O | None | 36.5 |
| 15 | 0.1 | 5 mL | 5.1 | 0.6 | H₂O | None | 15.9 |
| 16 | 0.1 | 5 mL | 17 | 1.9 | Died | Chicken | 46.0 |
| | | | | | H₂O | | |
| 17 | 0.1 | 5 mL | 10.2 | 1.2 | Died | Chicken | 37.0 |
| | | | | | H₂O | | |
| 18 | 0.1 | 5 mL | 5.1 | 0.6 | Died | Chicken | 16.9 |
| | | | | | H₂O | | |
| 19 | 0.05 | 3 mL | 17 | 1.0 | Air | None | 2.8 |
| 20 | 0.05 | 3 mL | 10.2 | 0.6 | Air | None | 2.7 |
| 21 | 0.05 | 3 mL | 5.1 | 0.3 | Air | None | 2.25 |
| 22 | 0.05 | 3 mL | 17 | 1.0 | H₂O | None | 18.25 |
| 23 | 0.05 | 3 mL | 10.2 | 0.6 | H₂O | None | 10.1 |
| 24 | 0.05 | 3 mL | 5.1 | 0.3 | H₂O | None | 5.0 |
| 25 | 0.05 | 3 mL | 17 | 1.0 | Died | Chicken | 24.4 |
| | | | | | H₂O | | |
| 26 | 0.05 | 3 mL | 10.2 | 0.6 | Died | Chicken | 12.9 |
| | | | | | H₂O | | |
| 27 | 0.05 | 3 mL | 5.1 | 0.3 | Died | Chicken | 7.6 |
| | | | | | H₂O | | |
| 28 | 0.05 | 5 mL | 17 | 1.9 | Air | None | 1.9 |
| 29 | 0.05 | 5 mL | 10.2 | 1.2 | Air | None | 1.2 |
| 30 | 0.05 | 5 mL | 5.1 | 0.6 | Air | None | 0.6 |
| 31 | 0.05 | 5 mL | 17 | 1.9 | H₂O | None | 47.0 |
| 32 | 0.05 | 5 mL | 10.2 | 1.2 | H₂O | None | 41.0 |
| 33 | 0.05 | 5 mL | 5.1 | 0.6 | H₂O | None | 18.2 |
| 34 | 0.05 | 5 mL | 17 | 1.9 | Died | Chicken | 42.0 |
| | | | | | H₂O | | |
| 35 | 0.05 | 5 mL | 10.2 | 1.2 | Died | Chicken | 47.0 |
| | | | | | H₂O | | |
| 36 | 0.05 | 5 mL | 5.1 | 0.6 | Died | Chicken | 23.0 |
| | | | | | H₂O | | |

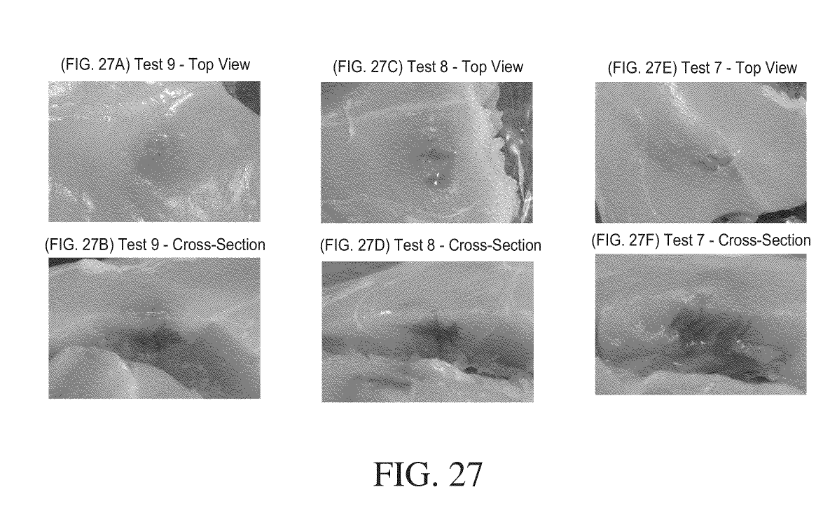

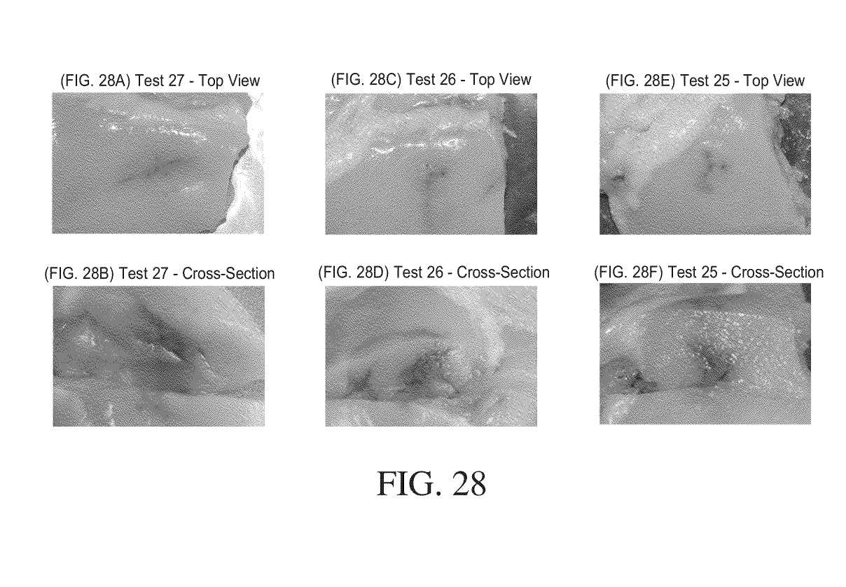

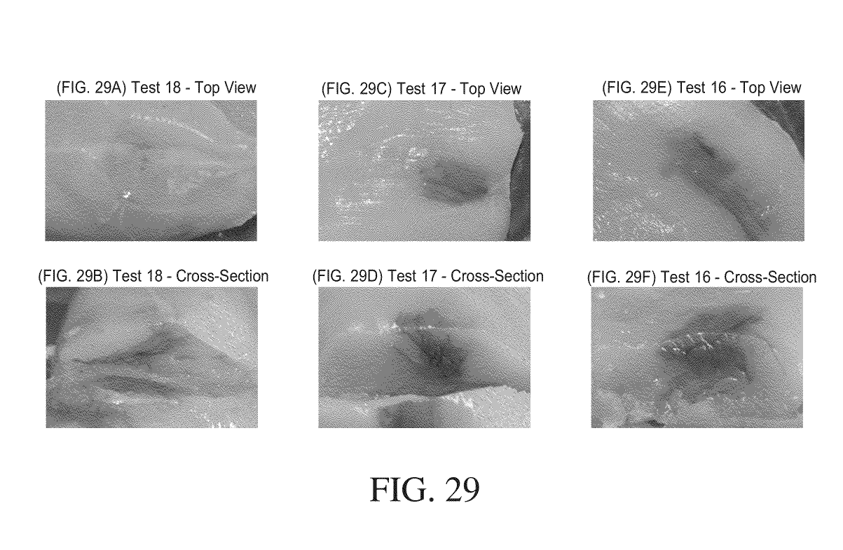

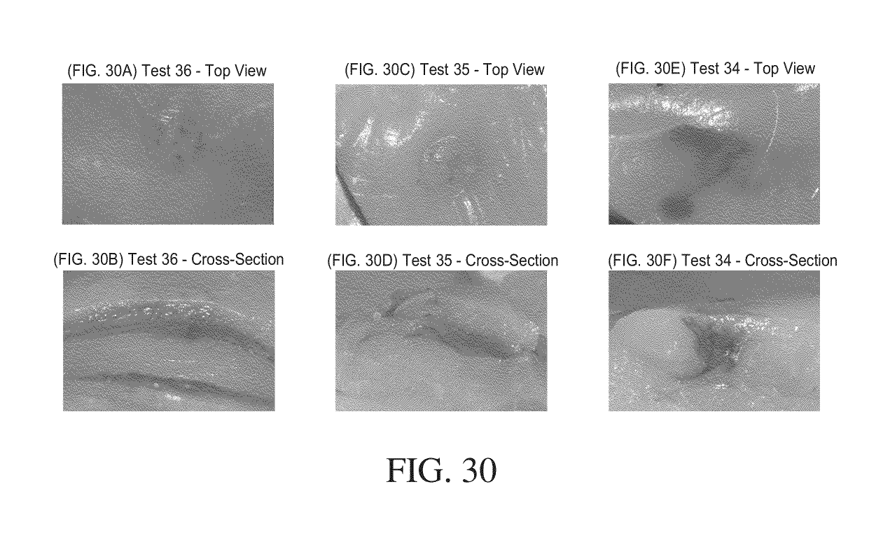

The spray patterns for water into an open area were studied using a high-speed camera. Generally, tests that produced a 1 mL/s flow rate or higher produced a well-defined, symmetric spray pattern that is expected to increase pressure and may be suitable for delivering therapeutic material. FIGS. 27-30 show top and cross-sectional views of chicken breast after injection with died water.

### Example 8

This example describes using the injection needles disclosed herein to inject material into a tissue sample by hand to consider the practical pressure limits for manually delivering material. The needles were configured the same is Example 7 and included 0.05 mm apertures with a 3 mm spacing between needles. The 3 mL syringe was supported using a support jig and the plunger was manually depressed as quickly as possible. The plunger motion was recorded using a high-speed camera and used to calculate the time for injecting 0.3 mL of died water into the chicken breast.

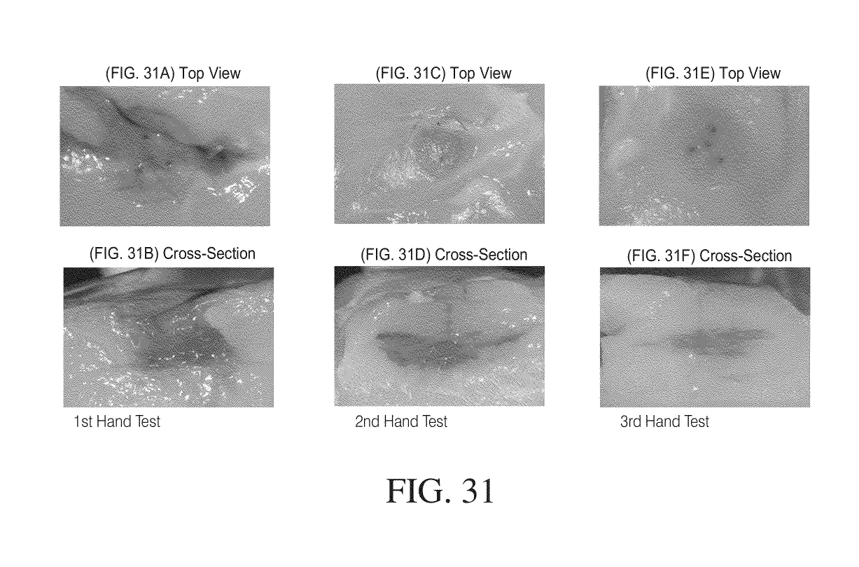

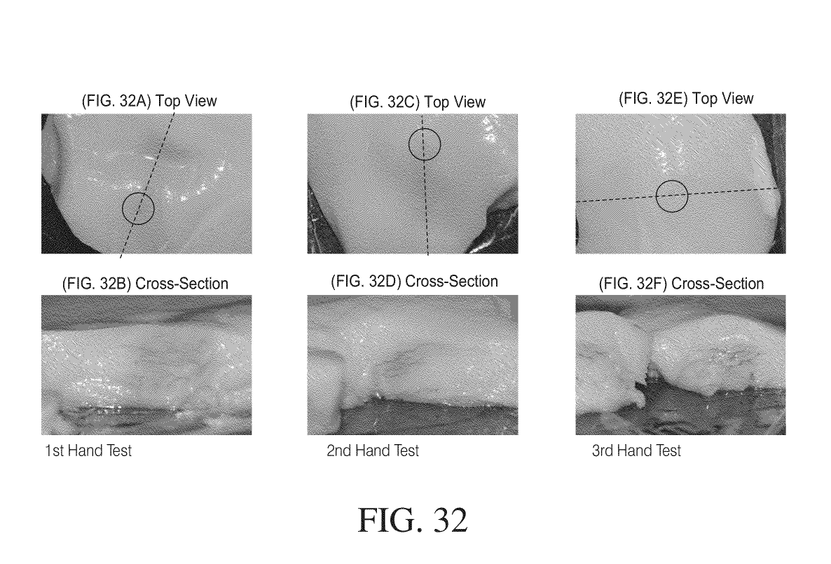

The test was repeated three times and the time required for delivering the material was 0.48 s, 0.40 s, and 0.48 s. Therefore, the average hand delivery speed was about 0.45 seconds. FIG. 31 shows top and cross-sectional views of chicken breast after manual injection with died water. FIG. 32 is a comparative example showing top and cross-sectional views of chicken breast after manual injection with died water using only a single needle.

What is claimed is:

1. A hypodermic needle assembly comprising a plurality of needles, wherein each needle comprises:
a lumen adapted for the passage of a therapeutic material,
a needle barrel that comprises a plurality of apertures on the length of the needle barrel, wherein said needle barrel has a closed-end,
wherein at least two needles of the hypodermic needle assembly have different positions of apertures; and
wherein said hypodermic needle assembly further comprises a connector (700) configured to join said plurality of needles to a pressure generation element.

2. The hypodermic needle assembly of claim 1, wherein said hypodermic needle assembly comprises a circular, diamond, or ovoid array of said needles.

3. The hypodermic needle assembly of claim 1, wherein said plurality of said needles is configured such that the apertures on the needle barrels face each other.

4. The hypodermic needle assembly of claim 1, wherein said plurality of said needles is configured such that all of the apertures are configured to oppose another aperture on a different needle.

5. The hypodermic needle assembly of claim 1, wherein said needle assembly further comprises a pressure generation element joined to said hypodermic needle assembly.

6. The hypodermic needle assembly of claim 5, wherein the pressure generation element is a syringe.

7. The hypodermic needle assembly of claim 1, wherein a needle barrel is disposed along the longitudinal axis of the device and said needle barrel comprises apertures that face away from the center or longitudinal axis of the device and additional needle barrels comprise apertures that face inward toward the center.

8. The hypodermic needle assembly of claim 1, wherein least two needles comprise a plurality of apertures that are configured to direct the pressurized agent towards the longitudinal axis of the device.

9. The hypodermic needle assembly of claim 1, further comprising control circuitry to generate an electric current or an electromagnetic field, whereby one or more needle barrels transmit the generated current or field into a tissue.

10. A method of using the hypodermic needle assembly of claim 1 to deliver a nucleic acid to a tissue comprising:
providing the hypodermic needle assembly of claim 1, wherein said hypodermic needle assembly has a nucleic acid within the lumen of said plurality of needles;
introducing said plurality of needles of said hypodermic needle assembly into a tissue; and
delivering said nucleic acid from said lumen of said plurality of needles into said tissue.

11. The method of claim 10, wherein said nucleic acid comprises a sequence that encodes a hepatitis C virus (HCV) or hepatitis B virus (HBV) antigen or both.

12. The method of claim 11, wherein said nucleic acid comprises a sequence that encodes an HCV NS3 antigen.

13. The method of claim 11, wherein said nucleic acid comprises a sequence that encodes an HBV core antigen.

14. The method of claim 11, wherein said nucleic acid comprises a sequence that encodes an HCV NS3 antigen and an HBV core antigen.


SEQUENCE LISTING (33 sequences)

SEQ ID NO: 1 - protein, 183 aa, Artificial Sequence
  HBCAg (amino acid seq)
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 2 - DNA, 552 bp, Hepatitis B
atggacatcgacccttataaagaatttggagctactgtggagttactctcgtttttgccctccgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 3 - DNA, 552 bp, Artificial Sequence
  HBCAg (codon optimized nt seq)
atggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 4 - protein, 18 aa, Artificial Sequence
  NS3/4A junction (amino acid sequence)
SADLEVVTSTWVLVGGVL

SEQ ID NO: 5 - DNA, 54 bp, Artificial Sequence
  NS3/4A junction (codon optimized nt seq)
agcgccgacctggaggtggtgaccagcacctgggtgctggtgggcggcgtgctg

SEQ ID NO: 6 - protein, 16 aa, Artificial Sequence
  NS4A/B junction (amino acid sequence)
DEMEECSQHLPYIEQG

SEQ ID NO: 7 - DNA, 48 bp, Artificial Sequence
  NS4A/B junction (nucleotide sequence)
gacgagatggaggagtgcagccagcacctgccctacatcgagcagggc

SEQ ID NO: 8 - protein, 869 aa, Artificial Sequence
  CONSTR-1 NS3/4A-HBcAg (NS3-NS4A-HBcAg fusion with active protease) (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTPTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 9 - DNA, 2610 bp, Artificial Sequence
  NS3/4A-HBcAg (NS3-NS4A-HBcAg fusion with active protease) (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgacccccacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 10 - protein, 869 aa, Artificial Sequence
  CONSTR-2 Mutant(catalytic triade)NS3/4A-HBcAg (NS3-NS4A-HBcAg fusion with inactive protease) (amino acid sequence)
MAPITAYAQQTRGLLGCIITSLTGRDKNQVEGEVQIVSTAAQTFLATCINGVCWTVYAGAGTRTIASPKGPVIQMYTNVDQALVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 11 - DNA, 2610 bp, Artificial Sequence
  Mutant(catalytic triade)NS3/4A-HBcAg (NS3-NS4A-HBcAg fusion with inactive protease) (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtacgccggcgccggcacccgcaccatcgccagccccaagggccccgtgatccagatgtacaccaacgtggaccaggccctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 12 - protein, 869 aa, Artificial Sequence
  CONSTR-3 NS3/4A-HBcAg (NS3 and NS4A-HBcAg fusion) (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 13 - DNA, 2610 bp, Artificial Sequence
  NS3/4A-HBcAg (NS3 and NS4A-HBcAg fusion) (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 14 - protein, 879 aa, Artificial Sequence
  CONSTR-4 NS3/4A-4Bjunct-HBcAg (NS3 AND NS4A AND HBcAg) (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 15 - DNA, 2640 bp, Artificial Sequence
  NS3/4A-4Bjunct-HBcAg (NS3 AND NS4A AND HBcAg) (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 16 - protein, 933 aa, Artificial Sequence
  CONSTR-5 NS3/4A-4Bjunct-HBcAg1-44-NS3/4Ajunct-HBc45-87-NS3/ 4Ajunct-HBc88-141-NS3/4Ajunct-HBc142-183 (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVSSADLEVVTSTWVLVGGVLYVNTNMGLKFRQLLWFHISCLTFGRETVIEYLVSFGVWIRTPPAYRPPNAPILSSADLEVVTSTWVLVGGVLTLPETTVVRRRGRSPRRRTPSPRRRRSQSPRRRRSQSRESQC

SEQ ID NO: 17 - DNA, 2802 bp, Artificial Sequence
  NS3/4A-4Bjunct-HBcAg1-44-NS3/4Ajunct- HBc45-87-NS3/4Ajunct-HBc88-141-NS3/4Ajunct-HBc142-183 (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 18 - protein, 933 aa, Artificial Sequence
  CONSTR-6 NS3/4A-4Bjunct-HBcAg142-183-NS3/4Ajunct-HBc45-87-N S3/4Ajunct-HBc88-141-NS3/4Ajunct-HBc1-44 (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLPEHCSPHHTALRQAILCWGELMTLATWVGVNLEDPASRDLVVSSADLEVVTSTWVLVGGVLYVNTNMGLKFRQLLWFHISCLTFGRETVIEYLVSFGVWIRTPPAYRPPNAPILSSADLEVVTSTWVLVGGVLMDIDPYKEFGATVELLSFLPSDFFPSVRDLLDTASALYREALES

SEQ ID NO: 19 - DNA, 2802 bp, Artificial Sequence
  NS3/4A-4Bjunct-HBcAg142-183-NS3/4Ajunct- HBc45-87-NS3/4Ajunct-HBc88-141-NS3/4Ajunct-HBc1-44 (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagctag

SEQ ID NO: 20 - protein, 933 aa, Artificial Sequence
  CONSTR-7 NS3/4A-4Bjunct-HBcAg142-183-NS3/4Ajunct-HBc88-141- NS3/4Ajunct-HBc45-87-NS3/4Ajunct-HBc1-44 (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLYVNTNMGLKFRQLLWFHISCLTFGRETVIEYLVSFGVWIRTPPAYRPPNAPILSSADLEVVTSTWVLVGGVLPEHCSPHHTALRQAILCWGELMTLATWVGVNLEDPASRDLVVSSADLEVVTSTWVLVGGVLMDIDPYKEFGATVELLSFLPSDFFPSVRDLLDTASALYREALES

SEQ ID NO: 21 - DNA, 2802 bp, Artificial Sequence
  NS3/4A-4Bjunct-HBcAg142-183-NS3/4Ajunct- HBc88-141-NS3/4Ajunct-HBc45-87-NS3/4Ajunct-HBc1-44 (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagctag

SEQ ID NO: 22 - protein, 933 aa, Artificial Sequence
  CONSTR-8 NS3/4A-4Bjunct-HBcAg142-183-NS3/4Ajunct-HBc88-141- NS3/4Ajunct-HBc1-44-NS3/4Ajunct-HBc45-87 (amino acid sequence)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLYVNTNMGLKFRQLLWFHISCLTFGRETVIEYLVSFGVWIRTPPAYRPPNAPILSSADLEVVTSTWVLVGGVLMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVS

SEQ ID NO: 23 - DNA, 2802 bp, Artificial Sequence
  NS3/4A-4Bjunct-HBcAg142-183-NS3/4Ajunct- HBc88-141-NS3/4Ajunct-HBc1-44-NS3/4Ajunct-HBc45-87 (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagctag

SEQ ID NO: 24 - protein, 933 aa, Artificial Sequence
  NS3/4A-4Bjunct-HBcAg142-183-NS3/4Ajunct- HBc88-141-NS3/4Ajunct-HBc1-44-NS3/4Ajunct-HBc45-87
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGYVNTNMGLKFRQLLWFHISCLTFGRETVIEYLVSFGVWIRTPPAYRPPNAPILSSADLEVVTSTWVLVGGVLTLPETTVVRRRGRSPRRRTPSPRRRRSQSPRRRRSQSRESQCSADLEVVTSTWVLVGGVLMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVS

SEQ ID NO: 25 - DNA, 2802 bp, Artificial Sequence
  NS3/4A-4Bjunct-HBcAg88-141-NS3/4Ajunct- HBc142-183-NS3/4Ajunct-HBc1-44-NS3/4Ajunct-HBc45-87 (nucleotide sequence)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagctag

SEQ ID NO: 26 - protein, 869 aa, Artificial Sequence; identical to SEQ ID NO: 8
  NS3/4A HBcAg Fusion Protein

SEQ ID NO: 27 - protein, 869 aa, Artificial Sequence; identical to SEQ ID NO: 10
  NS3/4A-HBcAg Fusion Protein

SEQ ID NO: 28 - protein, 869 aa, Artificial Sequence; identical to SEQ ID NO: 12
  NS3/4A-HBcAg Fusion Protein

SEQ ID NO: 29 - protein, 879 aa, Artificial Sequence; identical to SEQ ID NO: 14
  NS3/4A-HBcAg Fusion Protein

SEQ ID NO: 30 - protein, 933 aa, Artificial Sequence; identical to SEQ ID NO: 16
  NS3/4A-HBcAg Fusion Protein

SEQ ID NO: 31 - protein, 933 aa, Artificial Sequence; identical to SEQ ID NO: 18
  NS3/4A-HBcAg Fusion Protein

SEQ ID NO: 32 - DNA, 569 bp, Artificial Sequence
  Codon Optimized HBcAg
gaattcgcaccatggacatcgacccctacaaggagttcggcgccaccgtggagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgctagtctaga

SEQ ID NO: 33 - DNA, 20 bp, Artificial Sequence
  A CpG containing Oligonucleotide
tccatgacgttcctgacgtt